(19) United States
(12) Patent Application Publication
(10) Pub. No.: US 2025/0345531 A1
(43) Pub. Date: Nov. 13, 2025

(54) DUAL-CHAMBER SPRAY DEVICE

(71) Applicant: SaNOtize Research and Development Corp., Vancouver (CA)

(72) Inventors: Gilly Regev, Vancouver (CA); Christopher C. Miller, North Vancouver (CA); Nir Nagar, Ganey Tiqva (IL)

(73) Assignee: SaNOtize Research and Development Corp., Vancouver, BC (CA)

(21) Appl. No.: 18/026,819

(22) PCT Filed: Sep. 16, 2021
(86) PCT No.: PCT/IB2021/000633

§ 371 Date: Mar. 16, 2023

Related U.S. Application Data
(60) Provisional application No. 63/079,277, filed on Sep. 16, 2020.

(51) Int. Cl. A61M 11/00; B05B 11/00; B05B 11/10

(52) U.S. Cl. CPC A61M 11/007; B05B 11/0091; B05B 11/1084; A61M 2202/0275; B05B 1/3426; B05B 11/1025; B05B 11/1047

(57) ABSTRACT

Devices and associated methods for storing and dispensing a chemical solution or mixture are disclosed and described. In one embodiment, such a device can comprise a liquid container including a first chamber operable to retain a first liquid and a second chamber operable to retain a second liquid. In one embodiment, the spray device can comprise a first pump partially within the first chamber operable to aspirate and retain the first liquid, and a second pump partially within the second chamber operable to aspirate and retain the second liquid. In another embodiment, the spray device can comprise a plurality of mixing spaces fluidly coupled to the first pump and the second pump and operable to allow mixing of the first liquid and the second liquid. In another aspect, the spray device can comprise a nozzle fluidly coupled to the plurality of mixing spaces.

### PRIORITY DATA

This application claims the benefit of U.S. Provisional Application Ser. No. 63/079,277, filed on Sep. 16, 2020, which is incorporated herein by reference.

### FIELD OF THE INVENTION

The present invention relates generally to spray devices and associated methods. Accordingly, the present invention involves the mechanical arts field and the chemical arts field.

### BACKGROUND OF THE INVENTION

Many chemical compounds show promise for various applications or uses, but in reality remain unusable due to various challenges such as instability, difficulty in transport and administration, or for other reasons. One example of such a compound is nitric oxide (NO). NO is one of the principle oxides of nitrogen. Because it is a free radical, it has an unpaired electron and is highly reactive. In mammals, NO is a signaling molecule in many physiological and pathological processes. For example, NO is biosynthesized in-vivo from L-arginine, oxygen, and NADPH by various nitric oxide synthase enzymes and known as endothelium-derived relaxing factor (EDRF). This is at least in part due to the fact that blood vessel endothelium uses NO to signal the surrounding smooth muscle to relax, thus resulting in vasodilation. Accordingly, NO plays a role in conditions that benefit from vasodilation, such as erectile dysfunction and angina pectoris among others.

Because NO is a free radical, it is highly reactive and presents a significant challenge to store and administer for therapeutic purposes. For example, closed systems which administer NO gas directly from an airtight gas cylinder to a subject have been the primary mechanism of administering NO for respiratory or dermal therapy. Such systems are cumbersome and difficult to obtain and transport. Moreover, such systems typically require administration under the direct supervision of a medical professional due to the easy possibility of overdose. Therefore, systems and devices which allow easy transport, reduce or eliminate stability issues, and facilitate self-administration of NO by a subject continue to be sought.

### SUMMARY OF THE INVENTION

In one embodiment, a spray device can comprise a liquid container including a first chamber operable to retain a first liquid and a second chamber operable to retain a second liquid. In one aspect, the spray device can comprise a first pump partially within the first chamber operable to aspirate and retain the first liquid, and a second pump partially within the second chamber operable to aspirate and retain the second liquid. In another aspect, the spray device can comprise a plurality of mixing spaces fluidly coupled to the first pump and the second pump and operable to allow mixing of the first liquid and the second liquid. In another aspect, the spray device can comprise a nozzle fluidly coupled to the plurality of mixing spaces.

In another example, a spray device can comprise a liquid container including a first chamber operable to retain a first liquid and a second chamber operable to retain a second liquid. In one aspect, the spray device can include a first pump partially within the first chamber operable to aspirate and retain the first liquid, and a second pump partially within the second chamber operable to aspirate and retain the second liquid. In another aspect, the spray device can include a mixing space fluidly coupled to the first pump and the second pump and operable to allow mixing of the first liquid and the second liquid. In one aspect, the plurality of mixing spaces can be substantially empty between each actuation of the pumps. The spray device can further include a nozzle.

In another example, a spray device can comprise a liquid container including a plurality of chambers operable to retain a plurality of separate liquids. In one aspect the spray device can include a first pump partially within a first chamber operable to aspirate and retain a first liquid, and a second pump partially within a second chamber operable to aspirate and retain a second liquid. In another aspect, the spray device can include a plurality of mixing spaces fluidly coupled to the first pump and the second pump and operable to allow mixing of the first liquid and the second liquid. The spray device can further comprise a nozzle.

In another embodiment, a method of dispensing a solution can include providing an active pharmaceutical ingredient (API) precursor in a first chamber of a liquid container and an activator in a second chamber of a liquid container of a spray device. In one aspect, the method can include activating the API precursor by mixing the API precursor and the activator a plurality of times in the spray device. In another aspect, the method can include dispensing the solution from the spray device.

In another example, a method of activating a solution can include providing an active pharmaceutical ingredient (API) precursor-containing solution in a first chamber of a liquid container and an activator-containing solution in a second chamber of the liquid container of a spray device. In one aspect, the method can include activating the API precursor by mixing the API precursor-containing solution and the activator-containing solution upon an act of dispensing the mixed solution from the spray device.

In another example, a method of administering a treatment can include providing an active pharmaceutical ingredient (API) precursor-containing solution in a first chamber of a liquid container and an activator in a second chamber of the liquid container of a spray device. In one aspect, the method can include activating the API precursor by mixing the API precursor-containing solution and the activator-containing solution a plurality of times in the spray device. In another aspect, the method can include dispensing the mixed solution from the spray device to a treatment situs.

In another example, a method of administering a treatment can comprise providing an active pharmaceutical ingredient (API) precursor-containing solution in a first chamber of a liquid container and an activator in a second chamber of the liquid container of a spray device. In one aspect, the method can comprise activating the API precursor by mixing the API precursor-containing solution and the activator-containing solution upon an act of dispensing the mixed solution from the spray device to a treatment situs.

In another example, a method of providing a therapeutically effective amount of an unstable active pharmaceutical ingredient (API) can include providing an API precursor-containing solution in a first chamber of a liquid container of a spray device. In one aspect, the method can include providing an activator-containing solution in a second chamber of the liquid container of the spray device. In another aspect, the method can include activating the therapeutically effective amount of the API precursor by mixing the API precursor-containing solution and the activator-containing solution a plurality of times in the spray device. In another aspect, the method can include dispensing the mixed solution to a treatment situs.

In another embodiment, a method of manufacturing a product suitable for administering nitric oxide releasing solution (NORS) after an extended storage period can comprise providing a nitric oxide (NO) donor-containing solution in a first chamber of a liquid container of a spray device, and providing an activator-containing solution in a second chamber of the liquid container of the spray device. The method can further comprise activating the NORS by mixing the NO donor-containing solution and the activator-containing solution upon an act of dispensing the mixed solution from the spray device.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a side view of a dual-chamber spray device in accordance with an example embodiment.

FIG. 2 is an exploded view of a dual-chamber spray device in accordance with an example embodiment.

FIG. 3a is a perspective view of a dual-chamber spray device including an attached safety cap in accordance with an example embodiment.

FIG. 3b is a side cross-sectional view of a dual-chamber spray device including an attached safety cap and a front cross-sectional view of a dual-chamber spray device including an attached safety cap in accordance with an example embodiment.

FIG. 3c is a side cross-sectional view of a dual-chamber spray device including an attached safety cap in accordance with an example embodiment.

FIG. 4a is a side view of a dual-chamber spray device including an attached safety cap in accordance with an example embodiment.

FIG. 4b is a side cross-sectional view of a dual-chamber spray device including an attached safety cap in accordance with an example embodiment.

FIG. 4c is a side cross-sectional view of a dual-chamber spray device including an attached safety cap in accordance with an example embodiment.

FIG. 4d illustrates a side view of a dual-chamber spray device including an attached safety cap in accordance with an example embodiment.

FIG. 4e is a side cross-sectional view of a dual-chamber spray device including an attached safety cap in accordance with an example embodiment.

FIG. 4f is a side cross-sectional view of a dual-chamber spray device including an attached safety cap in accordance with an example embodiment.

FIG. 5a is a perspective view of a dual-chamber spray device in accordance with an example embodiment.

FIG. 5b illustrates a flow path in a dual-chamber spray device in accordance with an example embodiment.

FIG. 5c illustrates a flow path through a dual-chamber spray device in accordance with an example embodiment.

FIG. 6a is a side cross-sectional view of a pump of a dual-chamber spray device in accordance with an example embodiment.

FIG. 6b is a side cross-sectional view of a pump of a dual-chamber spray device in accordance with an example embodiment.

FIG. 7 is a side cross-sectional view of a dual-chamber spray device in accordance with an example embodiment.

FIG. 8a is a top view of a pump housing of a dual-chamber spray device in accordance with an example embodiment.

FIG. 8b is a perspective view of a dual-chamber spray device in accordance with an example embodiment.

FIG. 8c is a side view of a dual-chamber spray device in accordance with an example embodiment.

FIG. 9a is a top view of a gasket of a dual-chamber spray device in accordance with an example embodiment.

FIG. 9b is a perspective view of a gasket of a dual-chamber spray device in accordance with an example embodiment.

FIG. 9c is a side cross-sectional view of a gasket of a dual-chamber spray device in accordance with an example embodiment.

FIG. 10a is a side view of a top of a dual-chamber spray device in accordance with an example embodiment.

FIG. 10b is a perspective view of a top housing of a dual-chamber spray device in accordance with an example embodiment.

FIG. 10c is a top view of a top housing of a dual-chamber spray device in accordance with an example embodiment.

FIG. 11a is top view of a connecting plate of a dual-chamber spray device in accordance with an example embodiment.

FIG. 11b is side view of a connecting plate of a dual-chamber spray device in accordance with an example embodiment.

FIG. 11c is side cross-sectional view of a connecting plate of a dual-chamber spray device in accordance with an example embodiment.

FIG. 11d is perspective view of a connecting plate of a dual-chamber spray device in accordance with an example embodiment.

FIG. 12a is side view of a top of a dual-chamber spray device in accordance with an example embodiment.

FIG. 12b is perspective view of a top housing of a dual-chamber spray device in accordance with an example embodiment.

FIG. 12c is top view of a top housing of a dual-chamber spray device in accordance with an example embodiment.

FIG. 12d is side cross-sectional view of a top housing of a dual-chamber spray device in accordance with an example embodiment.

FIG. 13a is a top view of a post of a dual-chamber spray device in accordance with an example embodiment.

FIG. 13b is a side view of a post of a dual-chamber spray device in accordance with an example embodiment.

FIG. 13c is a perspective view of a post of a dual-chamber spray device in accordance with an example embodiment.

FIG. 13d is a top view of a post of a dual-chamber spray device in accordance with an example embodiment.

FIG. 14a is a perspective view of a dual-chamber spray device in accordance with an example embodiment.

FIG. 14b is a front view of a dual-chamber spray device in accordance with an example embodiment.

FIG. 14c is a side view of a dual-chamber spray device in accordance with an example embodiment.

FIG. 15 is a front view of a dual-chamber spray device in accordance with an example embodiment.

FIG. 16 is an exploded view of a dual-chamber spray device in accordance with an example embodiment.

FIG. 17a is front view of a dual-chamber spray device in accordance with an example embodiment.

FIG. 17b is an exploded view of a dual-chamber spray device in accordance with an example embodiment.

FIG. 18a is a three-dimensional render of a dual-chamber spray device in accordance with an example embodiment.

FIG. 18b is a three-dimensional render of a dual-chamber spray device in accordance with an example embodiment.

FIG. 18c is a three-dimensional render of a dual-chamber spray device in accordance with an example embodiment.

FIG. 19a is perspective view of a three-dimensional render of a dual-chamber spray device in accordance with an example embodiment.

FIG. 19b is perspective view of a three-dimensional render of a dual-chamber spray device in accordance with an example embodiment.

FIG. 20 illustrates a method of use of a dual-chamber spray device in accordance with an example embodiment.

FIG. 21 depicts a spray device in accordance with an example embodiment.

FIG. 22 depicts a spray device in accordance with an example embodiment.

FIG. 23 depicts a spray device in accordance with an example embodiment.

FIG. 24 depicts a method of a spray device in accordance with an example embodiment.

FIG. 25 depicts a method of a spray device in accordance with an example embodiment.

FIG. 26 depicts a method of a spray device in accordance with an example embodiment.

FIG. 27 depicts a method of a spray device in accordance with an example embodiment.

FIG. 28 depicts a method of a spray device in accordance with an example embodiment.

FIG. 29a illustrates a nasal spray device in accordance with an example embodiment.

FIG. 29b illustrates a spray device in accordance with an example embodiment.

FIG. 29c illustrates a throat spray device in accordance with an example embodiment.

These drawings are provided to illustrate various aspects of the invention and are not intended to be limiting of the scope in terms of dimensions, materials, configurations, arrangements or proportions unless otherwise limited by the claims.

### DETAILED DESCRIPTION OF THE INVENTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used in accordance with the definitions set forth below.

The singular forms “a,” “an,” and, “the” in the written description include express support for plural referents unless the context clearly dictates otherwise.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term, like “comprising” or “including,” in the written description it is understood that direct support should also be afforded to “consisting essentially of” language as well as “consisting of” language as if stated explicitly and vice versa.

The terms “first,” “second,” “third,” “fourth,” and the like in the written description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the written description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in a mechanical or nonmechanical manner. Objects or structures described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

Reference throughout the written description to “an example” means that a particular feature, structure, or characteristic described in connection with the example is included in at least one embodiment. Thus, appearances of the phrases “in an example” in various places throughout this specification are not necessarily all referring to the same embodiment.

Reference in the written description may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art. For example, composition or system that has or provides “increased” stability exhibits a higher degree of stability as compared to a different, yet comparable composition or system, or as compared to a composition or system known in the art. A number of factors can cause such increased stability, including composition ingredients, system components or structures, operation, etc.

The term “coupled,” as used herein, is defined as directly or indirectly connected. “Directly coupled” objects or structures are in physical contact and are attached. “Fluidly coupled” objects, structures, or components are in a sufficient relationship so as to allow movement or transfer of fluid from one of the objects, structures, or components to the other. Objects described herein as being “adjacent to” each other may be in physical contact with each other, in close proximity to each other, or in the same general region or area as each other, as appropriate for the context in which the phrase is used.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Description of Embodiments

An initial overview of technology embodiments is provided below and specific technology embodiments are then described in further detail. This initial summary is intended to aid readers in understanding the technology more quickly, but is not intended to identify key or essential features of the technology, nor is it intended to limit the scope of the claimed subject matter.

Nitric oxide (NO) and other gases can have an effect on biological systems that allows the gases to be useful for therapeutic purposes. However, it can be impractical to apply such gases in their gas state to the skin, mucosal membranes, or body cavities in a way that can facilitate such biological effects or impart a therapeutic effect because of the cumbersome nature of gas container and supply equipment. While it may be possible to generate therapeutic amounts of NO from a solution that includes a nitric oxide precursor and an activator of the nitric oxide precursor, once the components are brought into combination, the production of NO typically proceeds in an uncontrolled manner and NO generated can be lost due to its high reactivity with other elements and compounds before it is presented at an application situs (e.g. even while still inside a container). Furthermore, combining the nitric oxide precursor and the activator at the time of the treatment is tedious and may not be efficacious.

Still further, separating the nitric oxide precursor and the activator in separate portions of a single container with a single dispensing mechanism may not preserve the stability of the combination when the nitric oxide precursor and the activator are combined a long period of time before administration. In addition, even when the nitric oxide precursor and the activator is combined before treatment without degrading, adequate mixing of the nitric oxide precursor and the activator can be difficult. For example, mixing the nitric oxide precursor and the activator only once before administration of the compound may result in a compound that is not sufficiently mixed to exhaust all of the available reactants. Mixing the nitric oxide precursor and the activator can also be unsafe for an untrained user.

Therefore, it would be useful to deliver an unstable compound in a way that prevents its decomposition over time and adequately mixes the compound but also preserves a user-friendly delivery mechanism.

In one embodiment, a spray device can comprise a liquid container including a first chamber operable to retain a first liquid and a second chamber operable to retain a second liquid. In one aspect, the spray device can comprise a first pump partially within the first chamber operable to aspirate and retain the first liquid, and a second pump partially within the second chamber operable to aspirate and retain the second liquid. In another aspect, the spray device can comprise a plurality of mixing spaces fluidly coupled to the first pump and the second pump and operable to allow mixing of the first liquid and the second liquid. In another aspect, the spray device can comprise a nozzle fluidly coupled to the plurality of mixing spaces. In another aspect, the plurality of mixing spaces can be substantially empty between each actuation of the pumps.

In another embodiment, a method of dispensing a solution can include providing an active pharmaceutical ingredient (API) precursor in a first chamber of a liquid container and an activator in a second chamber of a liquid container of a spray device. In one aspect, the method can include activating the API precursor by mixing the API precursor and the activator a plurality of times in the spray device. In another aspect, the method can include dispensing the solution from the spray device.

In another example, a method of activating a solution can include providing an active pharmaceutical ingredient (API) precursor-containing solution in a first chamber of a liquid container and an activator-containing solution in a second chamber of the liquid container of a spray device. In one aspect, the method can include activating the API precursor by mixing the API precursor-containing solution and the activator-containing solution upon an act of dispensing the mixed solution from the spray device.

In another embodiment, a method of manufacturing a product suitable for administering nitric oxide releasing solution (NORS) after an extended storage period can comprise providing a nitric oxide (NO) donor-containing solution in a first chamber of a liquid container of a spray device, and providing an activator-containing solution in a second chamber of the liquid container of the spray device. The method can further comprise activating the NORS by mixing the NO donor-containing solution and the activator-containing solution upon an act of dispensing the mixed solution from the spray device.

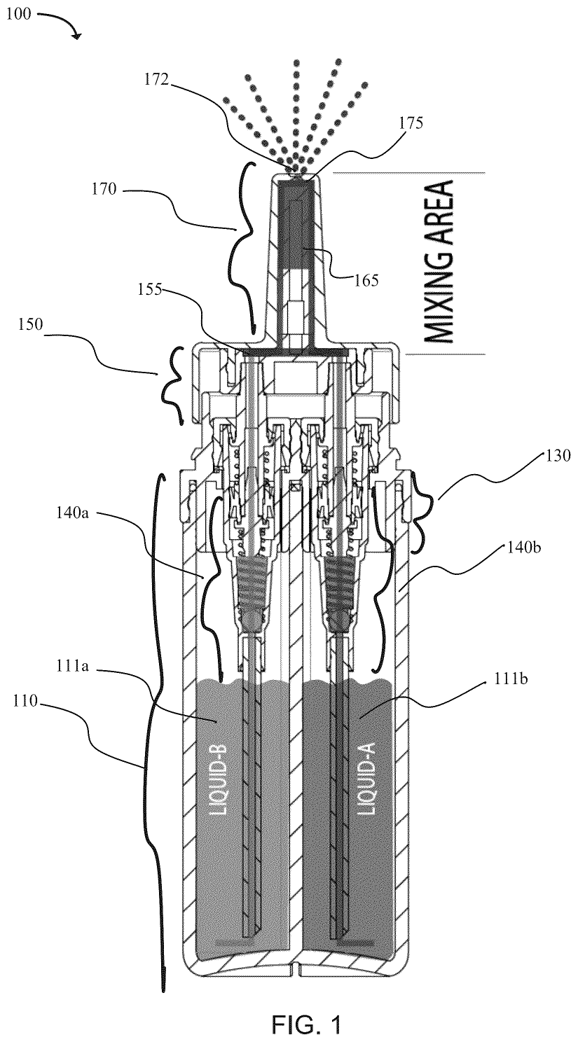

In one embodiment, as illustrated in FIG. 1, a spray device 100 can comprise a liquid container 110 including a first chamber 111a operable to retain a first liquid and a second chamber 111b operable to retain a second liquid. In one aspect, the spray device 100 can comprise a first pump 140a in fluid communication with (e.g. fully or partially within) the first chamber 111a operable to aspirate and retain the first liquid, and a second pump 140b in fluid communication with (e.g. fully or partially within) the second chamber 111b operable to aspirate and retain the second liquid.

In one example, the spray device 100 can comprise a plurality of mixing spaces 155, 165, 175 fluidly coupled to the first pump 140a and the second pump 140b and operable to allow mixing of the first liquid and the second liquid. In another aspect, the spray device 100 can comprise a nozzle 172 fluidly coupled to the plurality of mixing spaces 155, 165, 175.

In one example, the plurality of mixing spaces 155, 165, 175 can include a first mixing space 155, and a second mixing space 165 fluidly coupled between the first mixing space 155 and the nozzle 172. The first mixing space 155 can be fluidly coupled to a first outlet from the first pump 140a and a second outlet from the second pump 140b. The plurality of mixing spaces can further include a nozzle mixing space 175 comprising a vortex tip of the nozzle 172. In another example, the spray device 100 can further include a pump housing 130, a connecting plate 150, and a top housing 170.

In another example, the spray device 100 can comprise a mixing space 155 or 165 fluidly coupled to the first pump 140a and the second pump 140b and operable to allow mixing of the first liquid and the second liquid. In one aspect, the mixing space 155 or 165 can be substantially empty between each actuation of the pumps 140a and 140b. In another aspect, the mixing space 155 or 165 can be substantially empty between each actuation of the pumps 140a, 140b. The spray device can further comprise a nozzle 172 fluidly coupled to the mixing space 155 or 165.

In another example, the spray device 100 can comprise a liquid container 110 including a plurality of chambers operable to retain a plurality of separate liquids. The spray device 110 can comprise a first pump 140a partially within a first chamber 111a operable to aspirate and retain a first liquid, and a second pump 140b partially within a second chamber 111b operable to aspirate and retain a second liquid. The spray device 100 can comprise a plurality of mixing spaces 155, 165, 175 fluidly coupled to the first pump 140a and the second pump 140b and operable to allow mixing of the first liquid and the second liquid. The spray device 100 can comprise a nozzle 172. In one example, the number of chambers can be greater than or equal to 2. In another example, the number of pumps can be greater than or equal to 2.

In another embodiment, a method of dispensing a solution can include providing an active pharmaceutical ingredient (API) precursor in a first chamber 111a of a liquid container 110 and an activator in a second chamber 111b of a liquid container 110 of a spray device 100. In one aspect, the method can include activating the API precursor by mixing the API precursor and the activator a plurality of times in the spray device 100. In another aspect, the method can include dispensing the solution from the spray device 100.

In another example, a method of activating a solution can include providing an active pharmaceutical ingredient (API) precursor-containing solution in a first chamber 111a of a liquid container 110 and an activator-containing solution in a second chamber 111b of the liquid container 110 of a spray device 100. In one aspect, the method can further include activating the API precursor by mixing the API precursor-containing solution and the activator-containing solution upon an act of dispensing the mixed solution from the spray device 100.

In another example, a method of administering a treatment can include providing an active pharmaceutical ingredient (API) precursor-containing solution in a first chamber 111a of a liquid container 110 and an activator in a second chamber 111b of the liquid container 110 of a spray device 100. In one aspect, the method can include activating the API precursor by mixing the API precursor-containing solution and the activator-containing solution a plurality of times in the spray device 100. In another aspect, the method can include dispensing the mixed solution from the spray device 100 to a treatment situs.

In another example, a method of administering a treatment can include providing an active pharmaceutical ingredient (API) precursor-containing solution in a first chamber 111a of a liquid container 110 and an activator in a second chamber 111b of the liquid container 110 of a spray device 100. In one aspect, the method can include activating the API precursor by mixing the API precursor-containing solution and the activator-containing solution upon an act of dispensing the mixed solution from the spray device 100 to a treatment situs.

In another example, a method of providing a therapeutically effective amount of an unstable active pharmaceutical ingredient (API) can comprise providing an API precursor-containing solution in a first chamber 111a of a liquid container 110 of a spray device 100. In one aspect, the method can include providing an activator-containing solution in a second chamber 111b of the liquid container 110 of the spray device 100. In another aspect, the method can include activating the therapeutically effective amount of the API precursor by mixing the API precursor-containing solution and the activator-containing solution a plurality of times in the spray device 100. In another aspect, the method can include dispensing the mixed solution to a treatment situs.

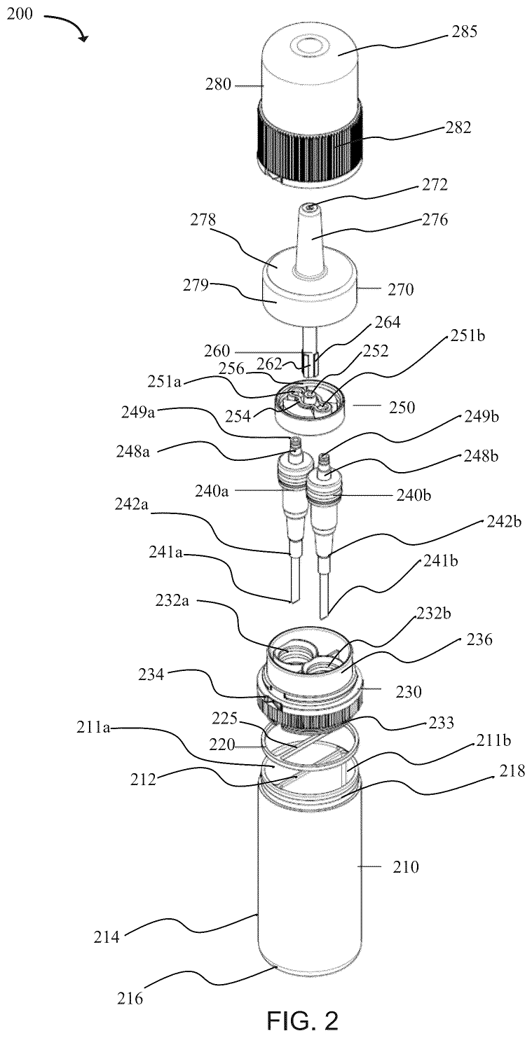

With this initial overview in mind, the spray devices of the current disclosure will be described in more detail. FIG. 2 illustrates an exploded view of a dual-chamber spray device 200 in accordance with an example. The dual-chamber spray device can include one or more of: a liquid container 210, a gasket 220, a pump housing 230, two or more pumps 240a and 240b, a connecting plate 250, a post 260, a top housing 270, or a cap 280.

In one example, the liquid container 210 can include a divider 212 that separates a first chamber 211a and a second chamber 211b. The first chamber 211a can be operable to retain a first liquid. The second chamber 211b can be operable to retain a second liquid. In one example, the volume of the first chamber 211a or the volume of the second chamber 211b can be from about 1 ml to about 100 ml. In one aspect, a ratio of a volume of the first chamber 211a and a volume of the second chamber 211b can be from about 1:10 to about 10:1. In another aspect, a volume of the first chamber 211a and a volume of the second chamber 211b can be substantially equal.

In one aspect, the liquid container 210 can include an outer housing 214 and a bottom 216 that forms a bottom of the first chamber 211a and the second chamber 211b. The liquid container 210 can include a connector end 218 that can be a connecting tongue that is operable to connect into a groove 233 in a base 234 of a pump housing 230. The liquid container 210 can comprise any suitable material including but not limited to food grade compatible or pharmaceutical compatible materials that satisfy the United States Pharmacopeia (USP) 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables.

In another example, the spray device can include a gasket 220. The gasket can be coupled between the liquid container 210 and a pump housing 230. The gasket can include a gasket divider 225. The gasket 220 can comprise any suitable material including but not limited to plastic, Teflon, rubber, silicone, nitrile, vinyl, neoprene, the like, and combinations thereof.

In another example, the spray device can include a pump housing 230. In one aspect, the pump housing 230 can be coupled between: the liquid container 210, an outlet tube 242a of a first pump 240a and an outlet tube 242b of the second pump 240b. In another aspect, the pump housing can include a first pump fitting 232a configured to retain the first pump 240a, and a second pump fitting 232b configured to retain the second pump 240b. In another aspect, the pump housing can include a base 234 including a groove 233 operable to connect to a connecting tongue 218 of the liquid container 210. In another aspect, the pump housing can include a collar 236 operable to connect to a connecting plate 250. The pump housing 230 can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables.

In another example, the spray device can include a first pump 240a and a second pump 240b. The first pump 240a can include a first inlet tube 242a, a first inlet 241a, a first outlet tube 248a, and a first outlet 249a. The first inlet tube 242a can be partially within the first chamber 211a. The first pump 240a can be partially within the first chamber 211a and operable to aspirate and retain the first liquid. The first inlet 241a can be within the first chamber 211a and operable to receive the first liquid from the first chamber 211a.

In one aspect, the first pump 240a or the second pump 240b can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables.

In one aspect, the first pump 240a can be operable to aspirate and retain a liquid amount of from about 0.01 ml to about 5 ml. In another aspect, the first pump 240a can be operable to aspirate and retain a liquid amount of from about 0.1 ml to about 2 ml. In another aspect, the first pump 240a can be operable to aspirate and retain a liquid amount of from about 0.5 ml to about 1 ml. In another aspect, the first pump 240a can be operable to aspirate and retain a liquid amount of from about 0.01 ml to about 0.50 ml.

In another example, the second pump 240b can include a second inlet tube 242b, a second inlet 241b, a second outlet tube 248b, and a second outlet 249b. The second inlet tube 242b can be partially within the second chamber 211b. The second pump 240b can be partially within the second chamber 211b and operable to aspirate and retain the second liquid. The second inlet 241b can be within the second chamber 211b and operable to receive the second liquid from the second chamber 211b.

In one aspect, the second pump 240b can be operable to aspirate and retain a liquid amount of from about 0.01 ml to about 5 ml. In another aspect, the second pump 240b can be operable to aspirate and retain a liquid amount of from about 0.1 ml to about 2 ml. In another aspect, the second pump 240b can be operable to aspirate and retain a liquid amount of from about 0.5 ml to about 1 ml. In another aspect, the second pump 240b can be operable to aspirate and retain a liquid amount of from about 0.01 ml to about 0.50 ml.

In one example, the viscosity of the first liquid of the second liquid, or a combination thereof can be from about 0.1 centipoise (cP) to about 10,000 cP. In another example, the viscosity of the first liquid of the second liquid, or a combination thereof can be from about 0.1 centipoise (cP) to about 1000 cP. In another example, the viscosity of the first liquid of the second liquid, or a combination thereof can be from about 0.1 centipoise (cP) to about 100 cP. In another example, the viscosity of the first liquid of the second liquid, or a combination thereof can be from about 0.1 centipoise (cP) to about 100 cP. In another example, the viscosity of the first liquid of the second liquid, or a combination thereof can be from about 0.1 centipoise (cP) to about 10 cP.

In one aspect, the spray device 200 can be operable to spray a liquid amount of from about 0.01 ml to about 5 ml upon each actuation of the first pump 240a or the second pump 240b. In another aspect, the spray device 200 can be operable to spray a liquid amount of from about 0.1 ml to about 2 ml upon each actuation of the first pump 240a or the second pump 240b. In another aspect, the spray device 200 can be operable to spray a liquid amount of from about 0.5 ml to about 1 ml upon each actuation of the first pump 240a or the second pump 240b. In another aspect, the spray device 200 can be operable to spray a liquid amount of from about 0.01 ml to about 0.50 ml upon each actuation of the first pump 240a or the second pump 240b.

In another example, the first pump 240a and the second pump 240b can be fluidly coupled to one or more mixing spaces to allow confluence or combination and/or mixing of the first liquid from the first pump 240a and the second liquid from the second pump 240b. In one aspect, the first outlet tube 248a can include a first outlet 249a that can be coupled to one or more mixing spaces. In another aspect, the second outlet tube 248b can include a second outlet 249b that can be coupled to one or more mixing spaces.

In one example, a ratio of a volume of: a capacity of the first pump 240a or the second pump 240b, and a volume of a first mixing space or a second mixing space can be from about 100:1 to about 1:1. In one example, the ratio of the volume of the capacity of the first pump 240a or the second pump 240b and a volume of the first mixing space can be from about 100:1 to about 1:1. In one example, the ratio of the volume of the capacity of the first pump 240a or the second pump 240b and a volume of the second mixing space can be from about 100:1 to about 1:1. In another example, a ratio of a volume of the first mixing space to a volume of the second mixing space can be from about 100:1 to about 1:1.

In another example, the spray device 200 can include a connecting plate 250 including a first inlet 251a, a second inlet 251b, a central abutment 252, an inner perimeter wall 254 defining at least a part of a first mixing space, and an outer perimeter wall 256. In one aspect, the connecting plate 250 can have a recess at least partially defining a mixing space and openings 251a and 251b fluidly coupling the first pump 240a and the second pump 240b to the mixing space. The connecting plate 250 can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables

In another example, the spray device 200 can comprise a post 260 including a plurality of nozzle grooves 262 and a plurality of channel-forming protrusions 264. In one aspect, the nozzle grooves 262 can fluidly couple different mixing spaces. In one example, the number of nozzle grooves 262 can be an integer greater than or equal to 1. In another example, the number of nozzle grooves can be an integer greater than or equal to 1. The post 260 can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables

In another example, the spray device 200 can include a top housing 270. The top housing can include one or more of a nozzle opening 272, a post cover 276, a post collar 278, or a sidewall 279. In one aspect, the top housing 270 can define a portion of a second mixing space and a portion of a nozzle mixing space. In one example, the nozzle opening 272 can be at a tip of the post cover 276. In another example, the nozzle opening 272 can be on a side of the post cover 276. The top housing 270 can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables

In one aspect, the nozzle opening 272 can be fluidly coupled to a plurality of mixing spaces (e.g., a first mixing space, a second mixing space, or a nozzle mixing space). In another aspect the plurality of mixing spaces can be substantially empty between each actuation of the pumps 240a and 240b.

In another example, a tip portion of the top housing can be configured to spray a liquid at an angle that can range from about 0° to about 90° relative to a flow path out of the second mixing space. In one example, when a spray device 200 is positioned upright on a surface, the second mixing space can have a direction of flow that is vertical with respect to the surface.

In this example, when the direction of flow out of the second mixing space is vertical, the tip portion of the top housing can be configured to spray the liquid at an angle of about 0° relative to the flow path out of the second mixing space (i.e. the direction of flow out of the second mixing space can be vertical and the direction of the spray from the tip portion of the top housing can also be vertical).

In another example, when the direction of flow out of the second mixing space is vertical, the tip portion of the top housing can be configured to spray the liquid at an angle of about 90° relative to the flow path out of the second mixing space (i.e. the direction of flow out of the second mixing space can be vertical and the direction of flow of the spray from the tip portion of the top housing can be perpendicular to the flow path out of the second mixing space).

In another example, a ratio of a volume of: a capacity of the first pump 240a or the second pump 240b, and a volume of a nozzle mixing space 275 can be from about 100,000:1 to about 10:1. In another example, a ratio of a volume of the first mixing space to a volume of the nozzle mixing space can be from about 10,000:1 to about 10:1. In another example, a ratio of a volume of the second mixing space to a volume of the nozzle mixing space can be from about 10,000:1 to about 10:1.

In another example, the nozzle opening 272 can be configured to spray a predetermined pattern of liquid including one or more of a hollow cone, a full cone, a spiral cone, a solid stream, a mist, a fog, a flat fan, the like, or combinations thereof. In another example, the liquid sprayed from the spray device can have an exit velocity from the nozzle opening 272 from about 6.5 m/s to about 19.5 m/s. In another example, the exit velocity can be from about 5 m/s to about 25 m/s. In another example, the exit velocity can be from about 13 m/s to about 19.5 m/s.

In another example, the spray device 200 can include a cap 280 that can be operable to be coupled to the top housing 270. The cap 280 can further include a cap base 282 and a cap collar 285. The cap 280 can comprise any suitable material including but not limited to food grade compatible or pharmaceutical compatible materials that satisfy the USP 661 standards such as high-density polyethylene (HDPE), low-density polyethylene (LDPE), polypropylene (PP), polyethylene terephthalate (PET), polyethylene terephthalate G (PETG), and plasticized polyvinyl chloride (PVC). Other suitable materials can comprise but are not limited to materials compatible with liquid or gels that comply with USP 1663 and USP 1664 for extractables/leachables.

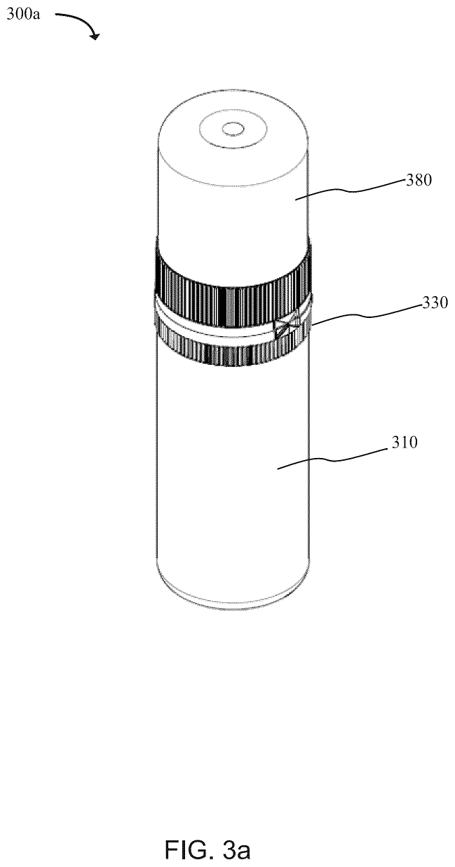

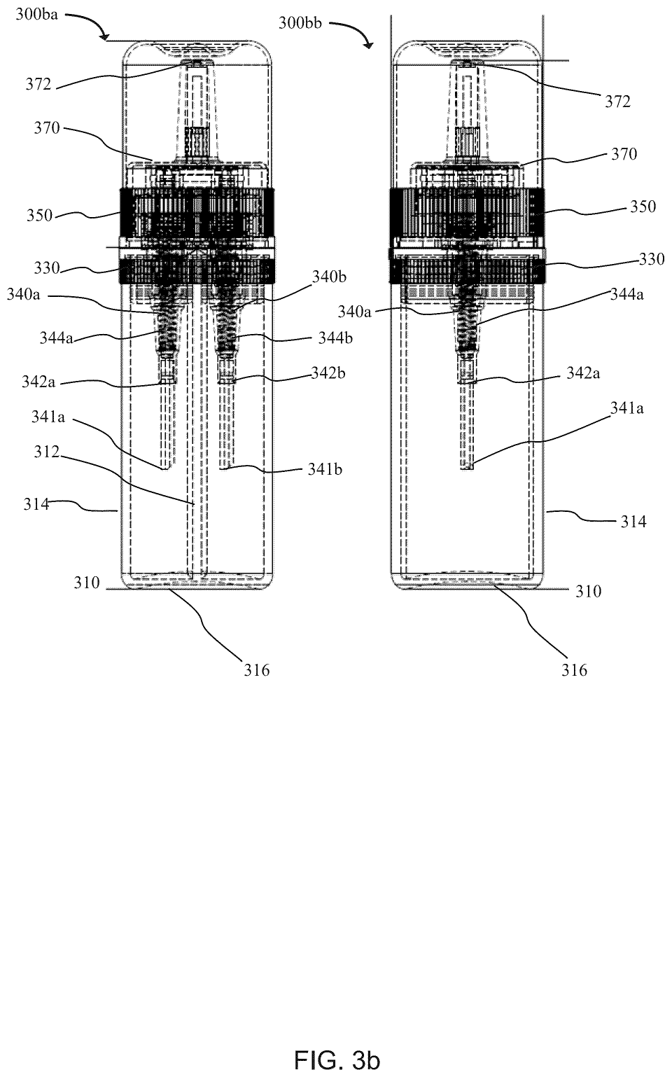

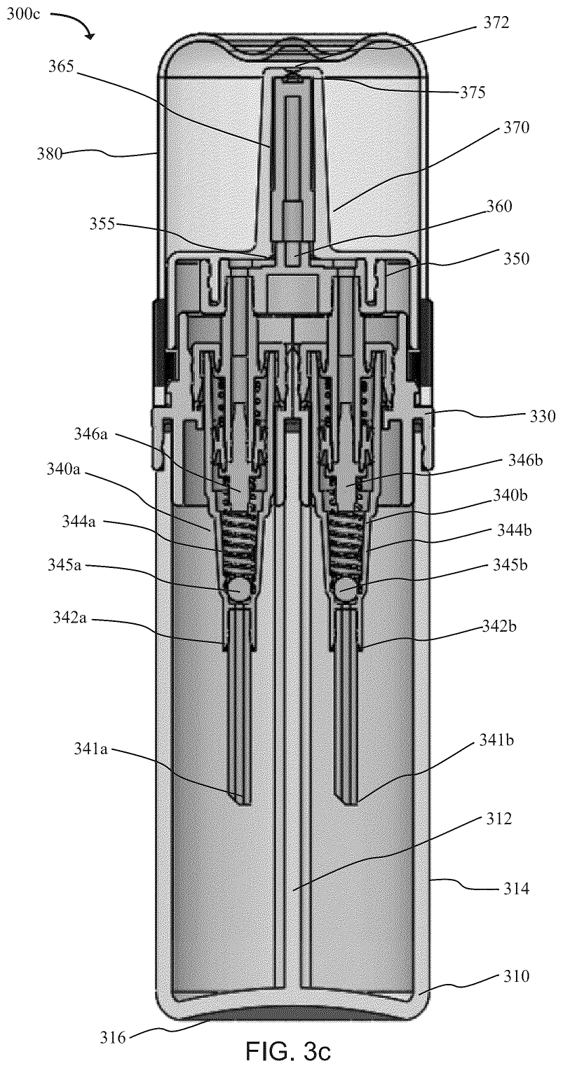

FIGS. 3a to 3c illustrates various perspectives of a dual-chamber spray device in accordance with an example. FIG. 3a is a perspective view of a dual-chamber spray device 300a including an attached safety cap 380 in accordance with an example. The spray device 300a can include a liquid container 310 and a base of a pump housing 330. In another example, FIG. 3b is a front cross-sectional view of a dual-chamber spray device 300ba and a side cross-section view of a dual-chamber spray device 300bb including an attached safety cap 380. In one aspect, as previously described, the dual-chamber spray device 300ba, 300bb can include a liquid container 310, a pump housing 330, a first pump 340a, a second pump 340b, a connecting plate 350, and a top housing 370 including a nozzle opening 372.

The liquid container 310 can include a divider 312 that forms a first chamber 311a and a second chamber 311b. The liquid container can further include an outer housing 314 and a bottom 316. In another aspect, the first pump 340a can include a first inlet 341a, a first inlet tube 342a, and a first spring 344a. In another aspect, the second pump 340b can include a second inlet 341b, a second inlet tube 342b, and a second spring 344b.

In another example, FIG. 3c illustrates a side cross-sectional view of a dual-chamber spray device 300c including an attached safety cap 380 in accordance with an example. The dual-chamber spray device 300c can include one or more of: a liquid container 310, a first pump 340a, a second pump 340b, a pump housing 330, a connecting plate 350, a post 360, a top housing 370, or a safety cap 380. In one aspect, the first pump 340a can include a first inlet tube 342a that can include a first inlet 341a, a first ball valve 345a, a first spring 344a, and a first piston 346a. In another aspect, the second pump 340b can include a second inlet tube 342b that can include a second inlet 341b, a second ball valve 345b, a second spring 344b, and a second piston 346b.

In this example, a plurality of mixing spaces can include two or more of a first mixing space 355 and a second mixing space 365, or a nozzle mixing space 375. The first mixing space 355 can be defined by a connecting plate 350 having a recess at least partially defining the first mixing space 355 and openings fluidly coupling the first pump 340a and the second pump 340b to the first mixing space 355. In this example, the second mixing space 365 can be fluidly coupled between the first mixing space 355 and the nozzle opening 372. In this example, the nozzle mixing space 375 can comprise a vortex tip of the nozzle. In one example, the plurality of mixing spaces can be substantially empty between each actuation of the pumps 340a and 340b.

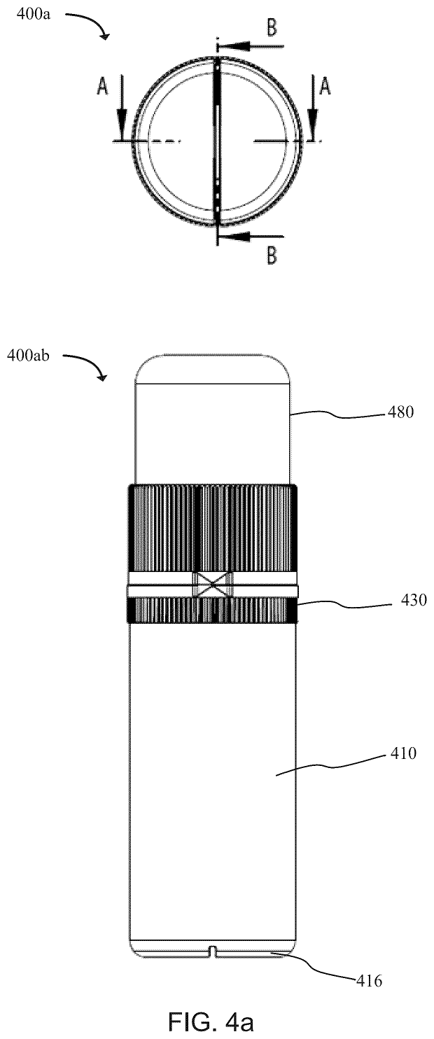

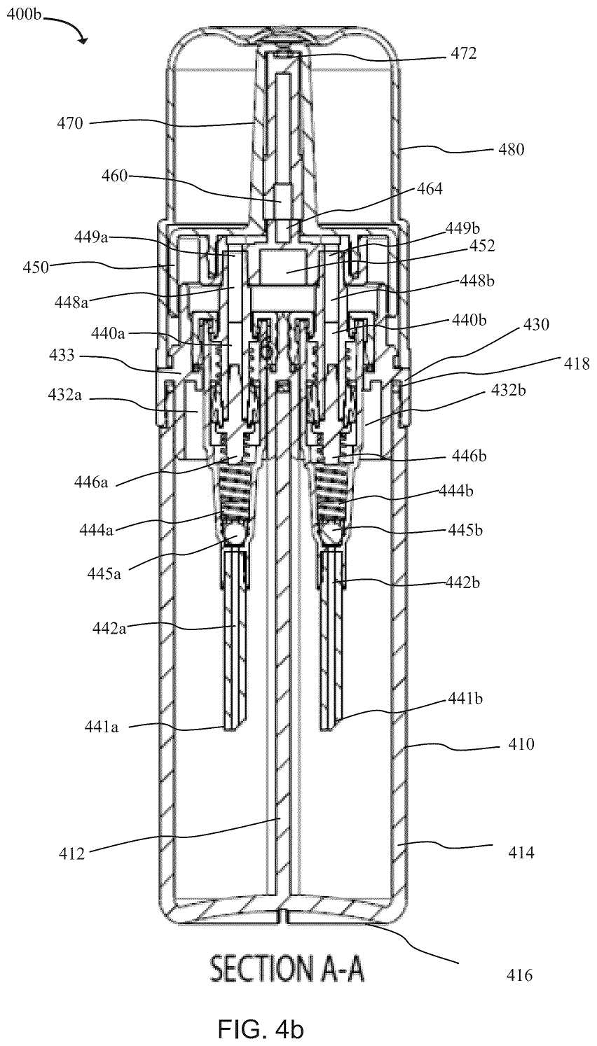

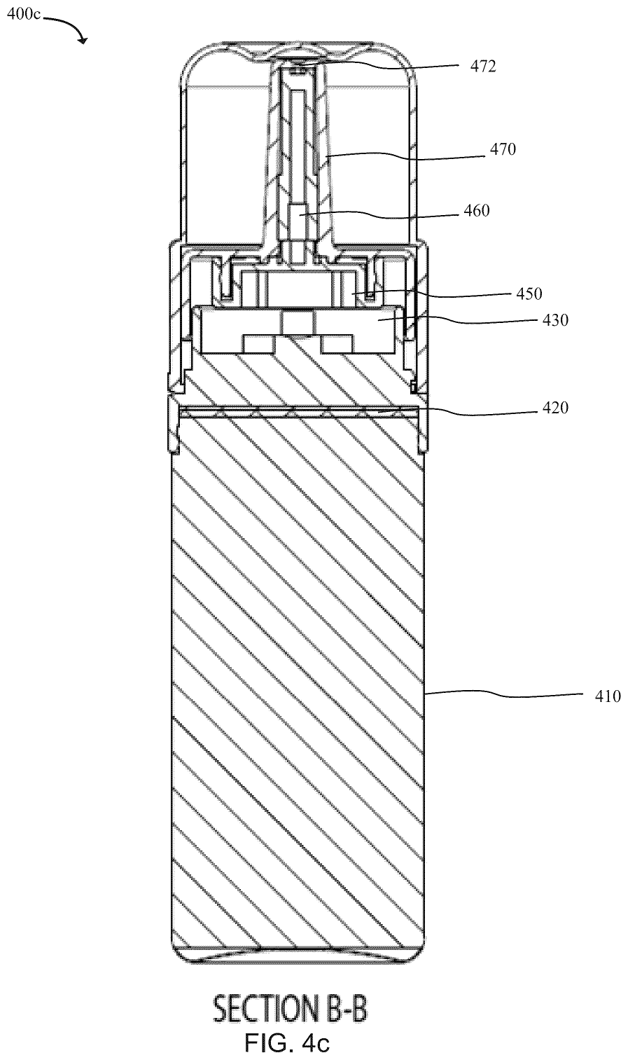

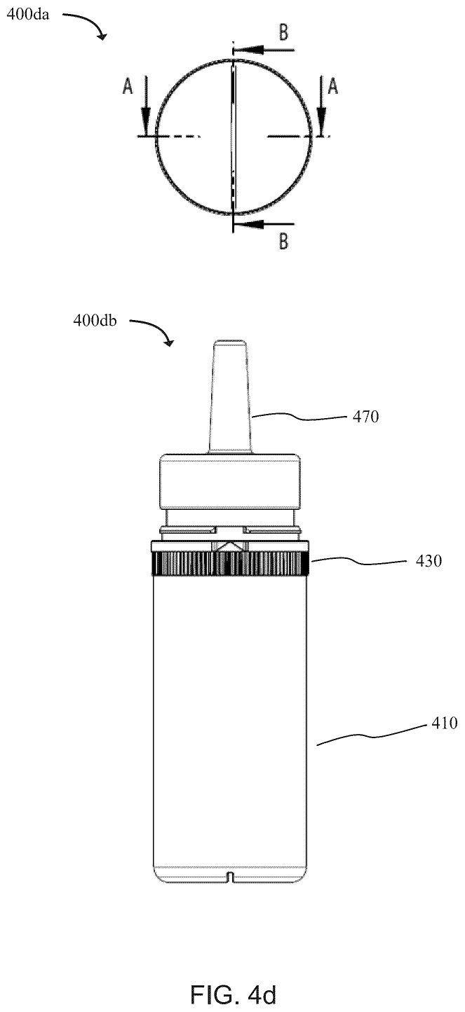

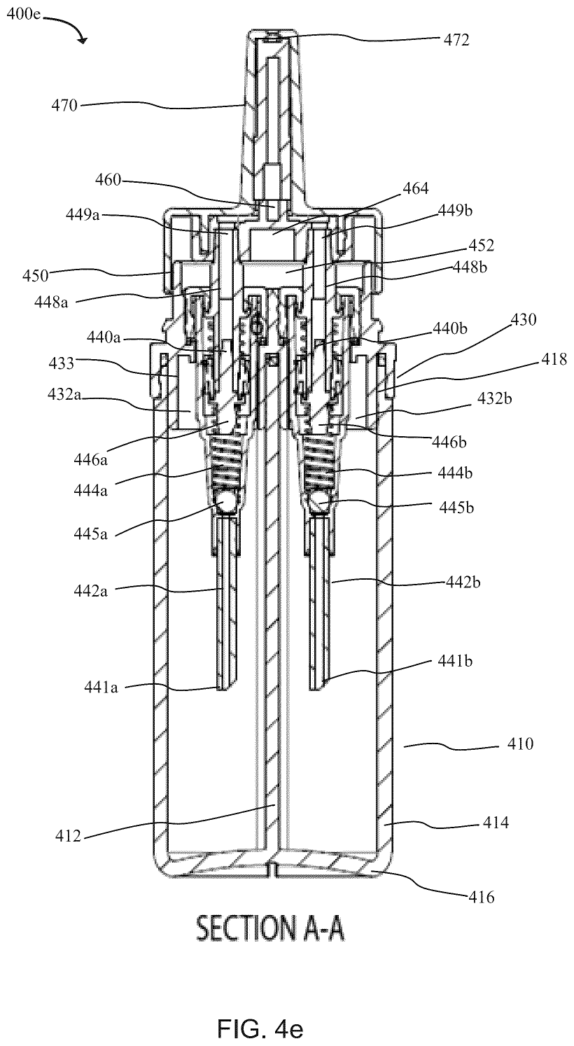

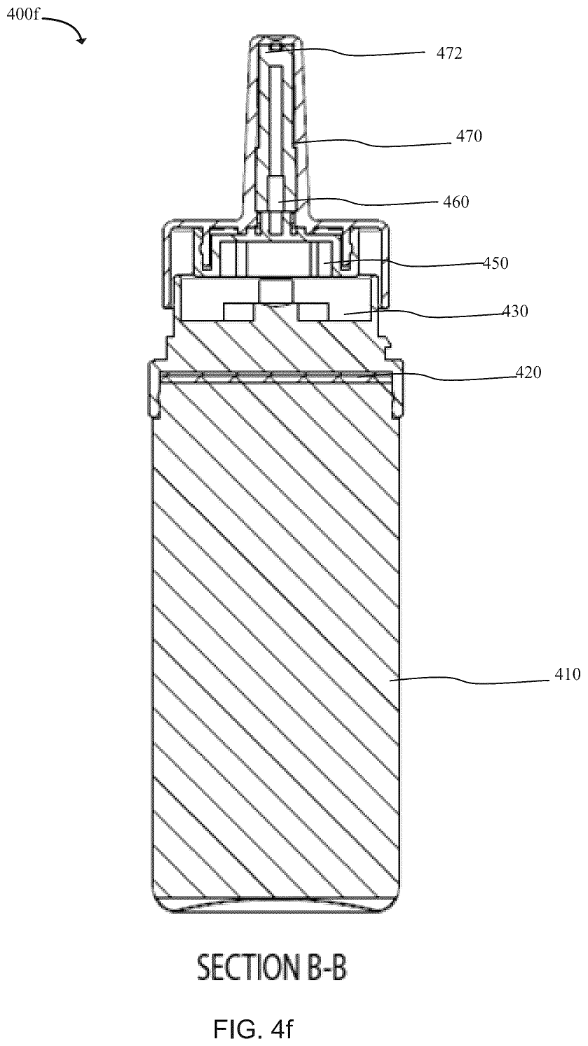

In another example, FIGS. 4a to 4f illustrate various views of a dual-chamber spray device. FIG. 4a illustrates a side view of a dual-chamber spray device 400a and 400ab including an attached safety cap in accordance with an example. The dual chamber spray device 400ab can include a safety cap 480, a base of a pump housing 430, and a liquid container 410 including a bottom 416.

In this example, as illustrated in FIG. 4b, a dual-chamber spray device 400b can include a liquid container with a divider 412, an outer housing 414, and a tongue 418. The first pump 440a can include a first inlet tube 442a that can include a first inlet 441a, a first ball valve 445a, a first spring 444a, and a first piston 446a. The second pump 440b can include a second inlet tube 442b that can include a second inlet 441b, a second ball valve 445b, a second spring 444b, and a second piston 446b. The first pump 440a can further include a first outlet tube 448a and a first outlet 449a. The second pump 440b can further include a second outlet tube 448ba nd a second outlet 449b.

In this example, the dual-chamber spray device 400b can include a pump housing 430. The pump housing 430 can include a first pump fitting 432a, a second pump fitting 432b, and a base groove 433. The first pump fitting 432a can be shaped and sized to hold the first pump 440a and the second pump fitting 432b can be shaped and sized to hold the second pump 440b.

In this example, the dual-chamber spray device 400b can further include a connecting plate 450. The connecting plate 450 can include a central abutment 452. The dual-chamber spray device 400b can further include a post 460 that can include one or more protrusions 464.

FIG. 4c illustrates a side cross-sectional view of a dual-chamber spray device 400c including an attached safety cap in accordance with an example. The dual-chamber spray device 400c can include a gasket 420.

In various examples, the dual-chamber spray device may not include a safety cap, as illustrated in FIGS. 4d to 4f. In one example, FIG. 4d illustrates a side view of a dual-chamber spray device 400da and 400 db that does not include an attached safety cap in accordance with an example. In one example, FIG. 4e illustrates a side cross-sectional view of the dual-chamber spray device 400e not including an attached safety cap in accordance with an example. In one example, FIG. 4f illustrates a side cross-sectional view of a dual-chamber spray device 400f not including an attached safety cap in accordance with an example.

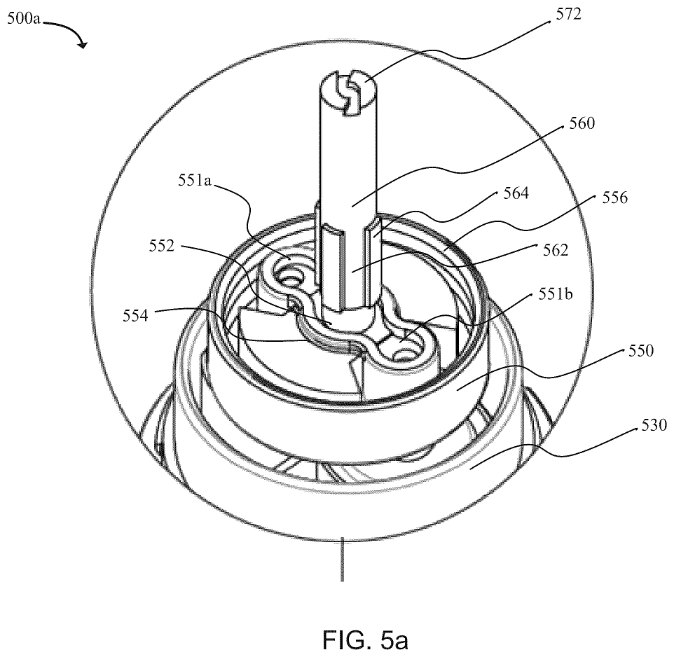

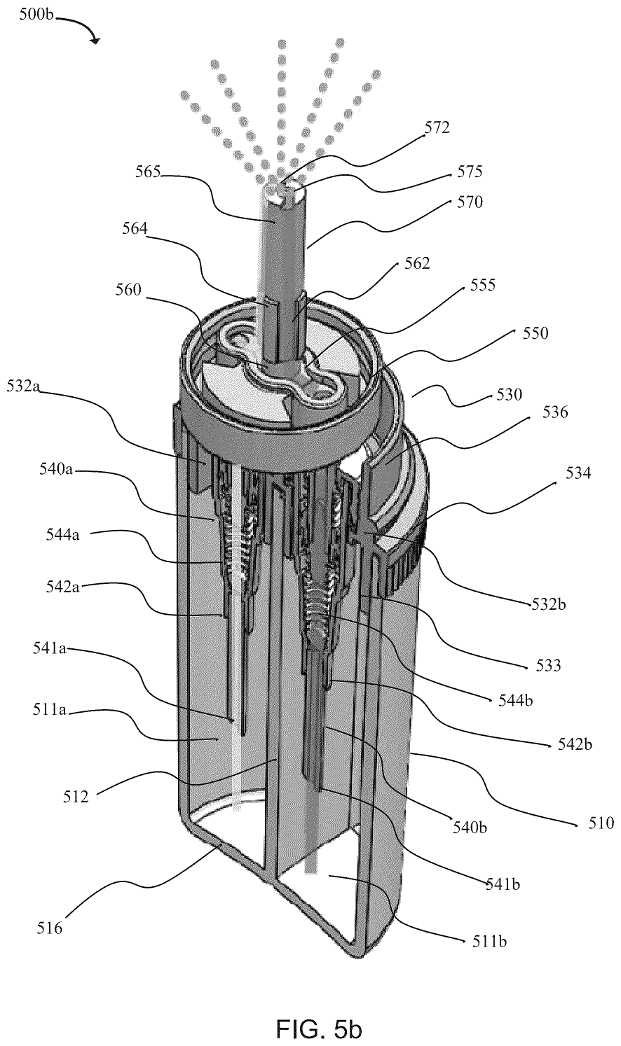

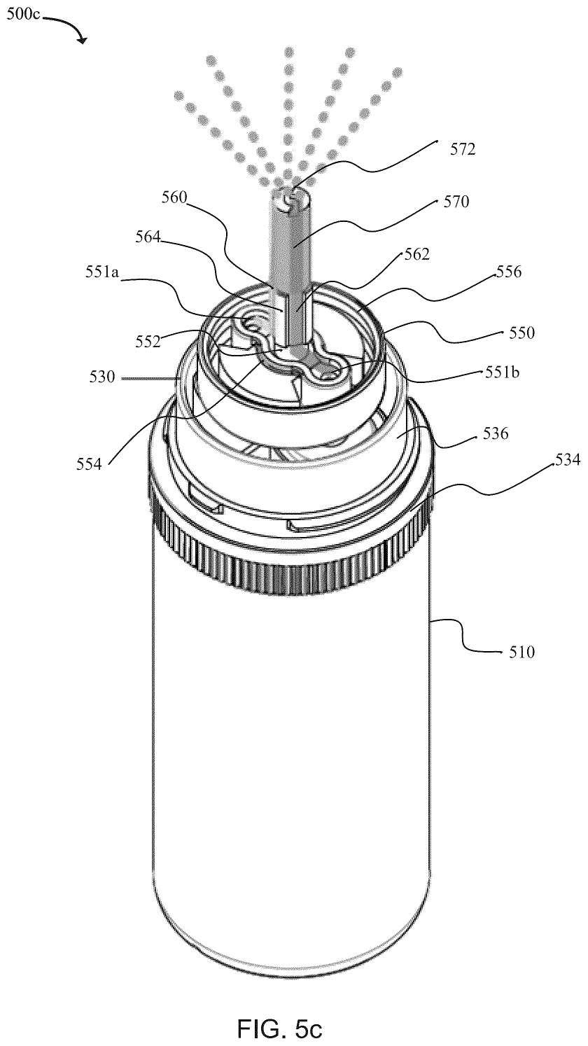

In various example, FIGS. 5a to 5c illustrate a post 560 of the dual-chamber spray device. FIG. 5a is a perspective view of a post 560 of a dual-chamber spray device 500a in accordance with an example. The post can include a plurality of channel-forming protrusions 564 that can form a plurality of channels 562 or grooves 562. The top of the post can be coupled to a nozzle opening 572. The post can abut a central abutment 552 of a connecting plate 550. The connecting plate can include an inner perimeter wall 554 that can partially define a perimeter of the first mixing area. The connecting plate can include an outer perimeter wall 556. The connecting plate can include a first fluid inlet 551a and a second fluid inlet 551b. The first fluid inlet can be fluidly coupled to a first outlet tube of a first pump and the second fluid inlet can be fluidly coupled to a second outlet tube of a second pump. The dual-chamber spray device 500a can further include a pump housing 530.

In one example, FIG. 5b illustrates a flow path in a dual-chamber spray device 500b. In this example, a first liquid can flow from a first chamber 511a of a liquid container 510 to a first inlet 541a of a first pump 540a. The first chamber 511a can be bounded by a bottom 516, a divider 512, an outer housing, and a gasket. The first pump 540a can include a first inlet 541a, a first inlet tube 542a, and a first spring 544a. The second pump 540b can include a second inlet 541b, a second inlet tube 542b, and a second spring 544b. The first pump 540a and the second pump 540b can be held in position by a first pump fitting 532a and a second pump fitting 532b of a pump housing 530. The pump housing can include a base 534 and a collar 536.

In one aspect, from the first inlet 541a, the first liquid can flow through the first inlet tube 542a to the first spring 544a when the ball-valve 545a is displaced upward by a dispensing event. When an unused dual-chamber spray device 500b initially has a dispensing event, the springs 544a can be empty. Upon the initial dispensing event, a piston can displace the springs 544a downward to create a vacuum that can displace the ball-valve 545a upward. When the ball-valve 545a is displaced upward, the first liquid can flow from the first inlet tube to the springs 544a until the ball-valve 545a is displaced downward to its original position blocking the first inlet tube 542a from directing additional liquid to the first spring 544a.

When the dual-chamber spray device 500b has previously had a dispensing event, the springs 544a can retain the first liquid. Upon an additional dispensing event, the first liquid can flow upward through the springs into an outlet tube of the first pump 540a and through a first outlet. The first liquid can exit the first outlet into a first mixing space 555 formed partially from an inner perimeter wall in the connecting plate 550.

In another aspect, from the second inlet 541b, the second liquid can flow through the second inlet tube 542b to the second spring 544b when the ball-valve 545b is displaced upward by a dispensing event. When an unused dual-chamber spray device 500b initially has a dispensing event, the spring 544b can be empty. Upon the initial dispensing event, a piston can displace the spring 544b downward to create a vacuum that can displace the ball-valve 545b upward. When the ball-valve 545b is displaced upward, the second liquid can flow from the second inlet tube to the second spring 544b until the ball-valve 545b is displaced downward to its original position blocking the second inlet tube 542b from directing additional liquid to the second spring 544b.

When the dual-chamber spray device 500b has previously had a dispensing event, the second spring 544b can retain the second liquid. Upon an additional dispensing event, the second liquid can flow upward through the second spring 544b into a second outlet tube of the second pump 540b and through a second outlet. The second liquid can exit the second outlet into the first mixing space 555.

In one example, when the first liquid and the second liquid have both entered the first mixing space 555, the first and second liquid can mix initially to form a mixed solution. In one aspect, the mixed solution can flow from the first mixing space 555 through a plurality of nozzle grooves 562 formed in the post 560 by the channel-forming protrusions 564. In one aspect, upon exiting the plurality of nozzle grooves 562, the mixed solution can mix in a second mixing space 565 in the top housing 570. In one aspect, after exiting the second mixing space 565, the mixed solution can enter a nozzle mixing space 575. From the nozzle mixing space 575, the mixed solution can be sprayed from the dual-chamber spray device 500b.

In one example, FIG. 5c illustrates a flow path through a dual-chamber spray device 500c in accordance with an example. The dual-chamber spray device 500c comprises a liquid container 510, a pump housing 530, a connecting plate 550, a post 560, and a top housing 570. The pump housing 530 includes a base 534 and a collar 536. The connecting plate 550 includes an inner perimeter wall 554, an outer perimeter wall 556, a central abutment 552, a first fluid inlet 551a that can be fluidly coupled to a first pump and a second fluid inlet 551b that can be fluidly coupled to a second pump.

In one example, a first liquid from the first pump and a second liquid from the second pump can conjoin in a first mixing space 555. From the first mixing space 555, the mixed solution can be directed through nozzle grooves 562 formed from channel-forming protrusions 564 on a post 560. Upon exiting the nozzle grooves 562, the mixed solution can mix in a second mixing space 565 that can be partially contained within the top housing 570 that can include a post cover, a post collar, and a sidewall. Upon exiting the second mixing space 565, the mixed solution can be directed upward to a tip of a top housing 570 proximate the nozzle opening 572. The tip of the top housing 570 can include a third mixing space 575 in which the mixed liquid can mix before exiting from the spray device 500c.

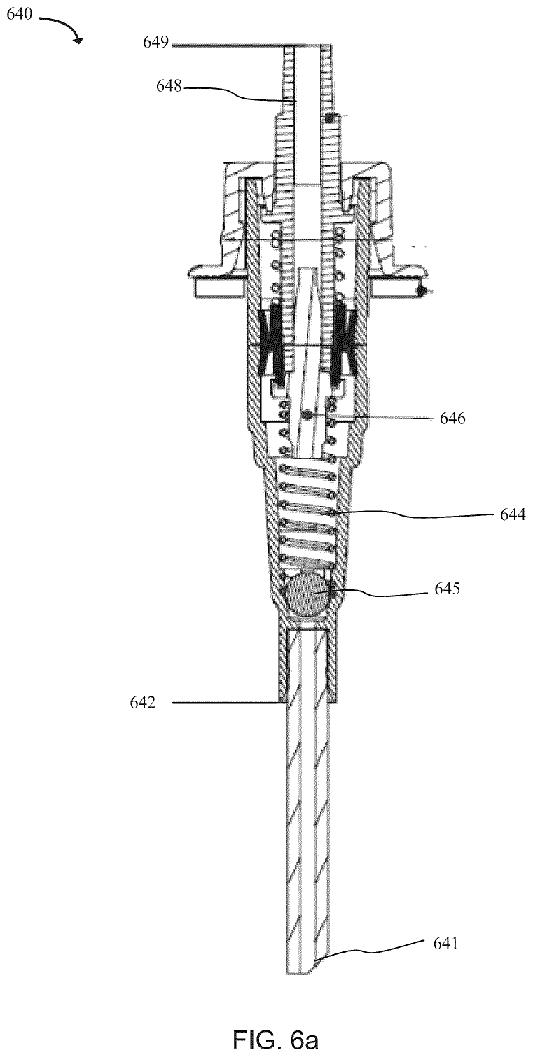

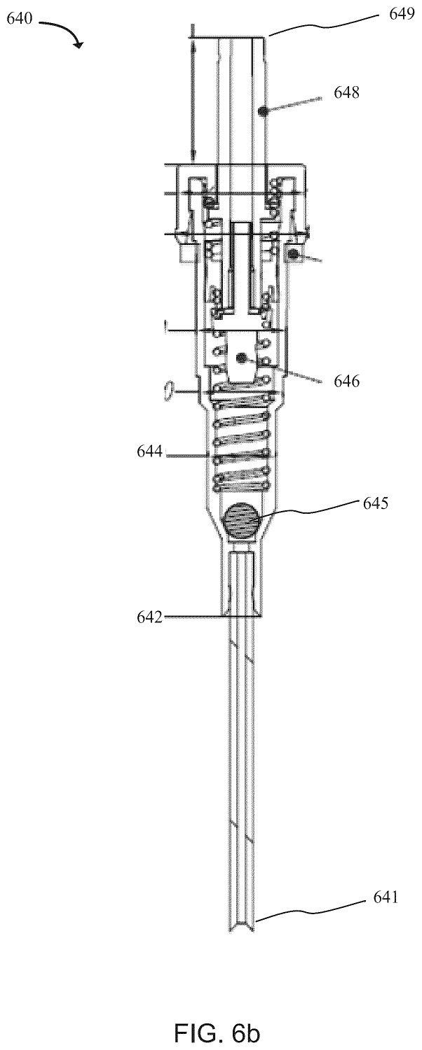

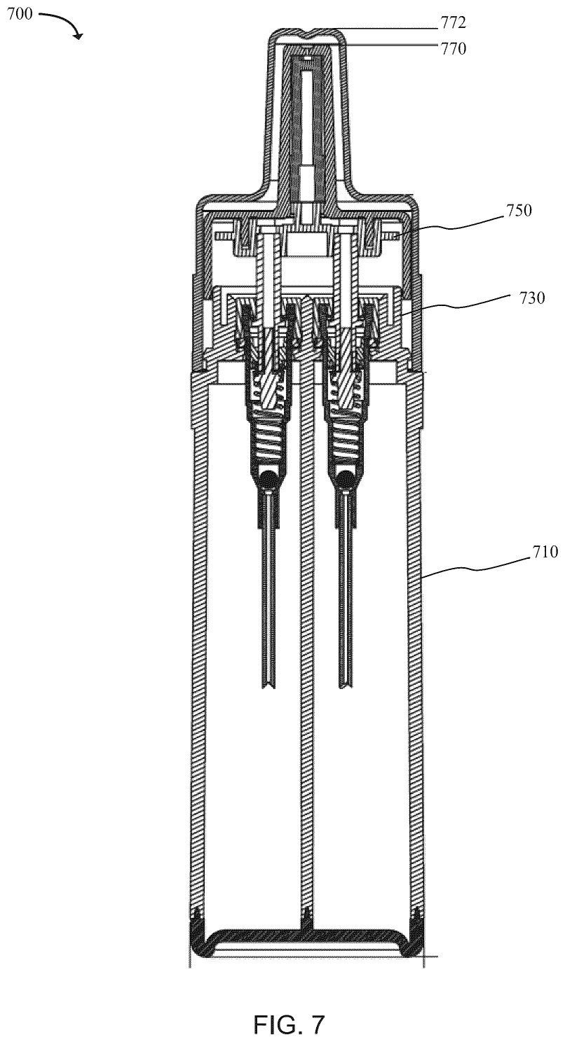

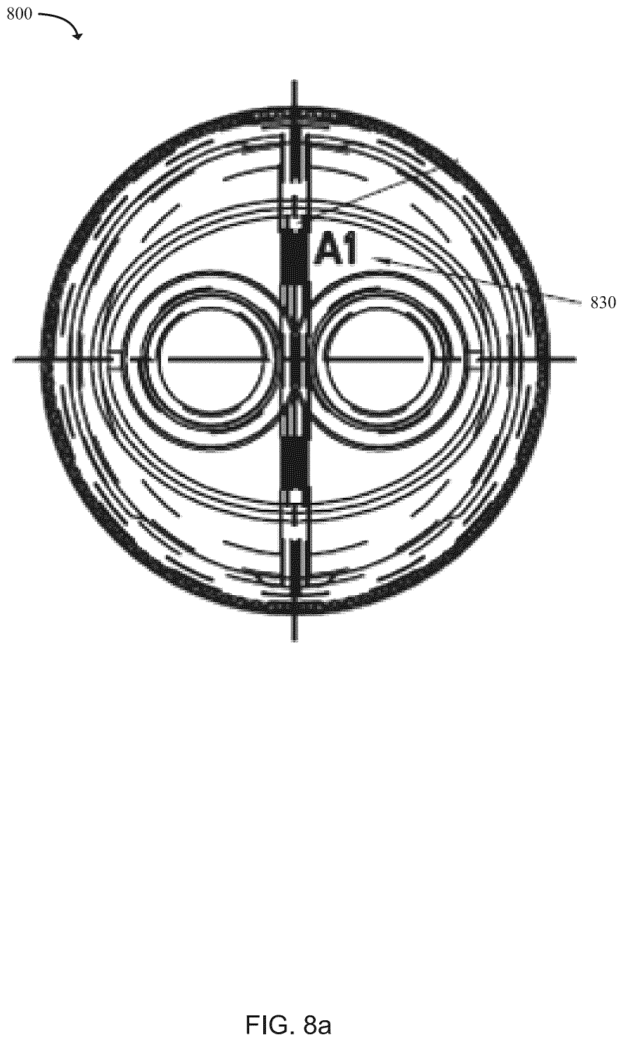

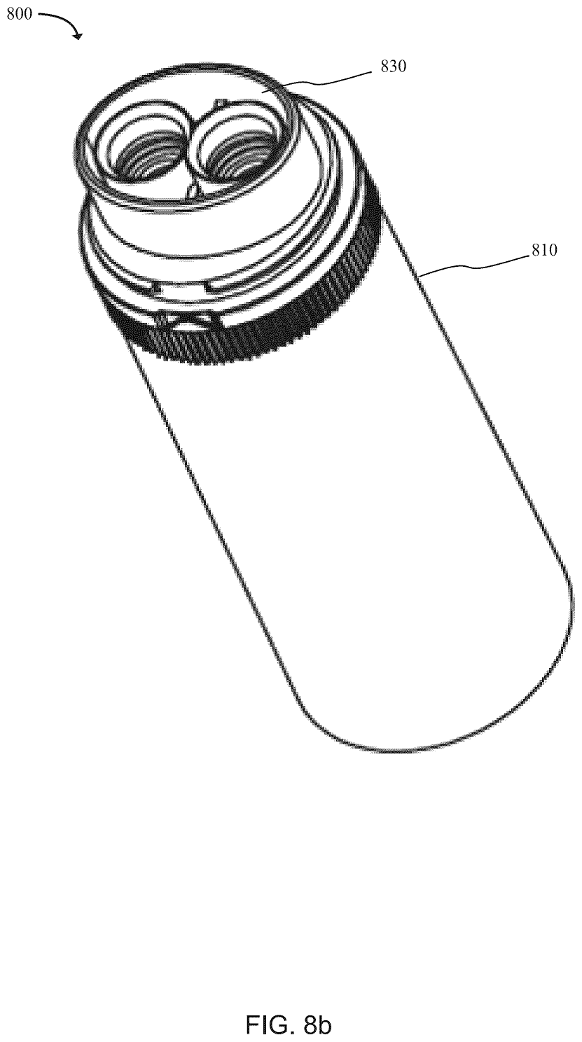

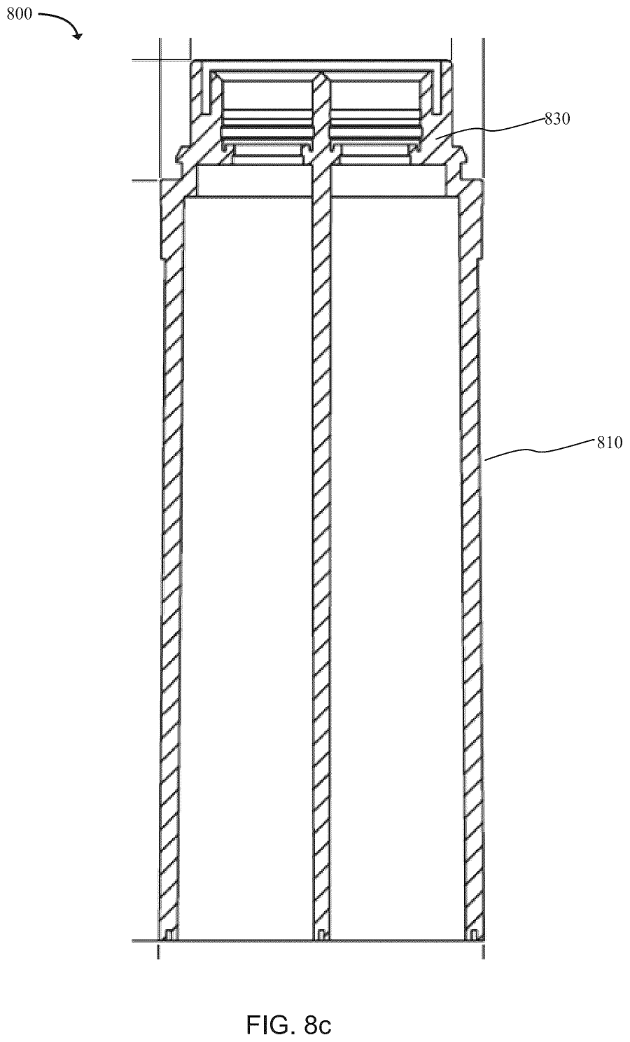

In another example, FIGS. 6a and 6b illustrate a pump 640. In one example, as illustrated in the cross-section in FIG. 6a, a pump 640 can include one or more of an inlet 641, an inlet tube 642, a spring 644, a ball valve 645 a piston 646, an outlet tube 648, or an outlet 649. In another example, as illustrated in the cross-section in FIG. 6b, a pump 640 can include one or more of an inlet 641, an inlet tube 642, a spring 644, a ball valve 645 a piston 646, an outlet tube 648, or an outlet 649. In one example, FIG. 7 is a side cross-sectional view of a dual-chamber spray device 700 in accordance with an example. In this example, the dual-chamber spray device 700 can include a liquid container 710, a pump housing 730, a connecting plate 750, a top housing 770, and a nozzle opening 772. In one example, FIG. 8a is a top view of a pump housing 830 of a dual-chamber spray device 800 in accordance with an example. FIG. 8b is a perspective view of a dual-chamber spray device 800 in accordance with an example. The dual-chamber spray device can include a liquid container 810 that can be fluidly coupled to the pump housing 830. FIG. 8c illustrates a side view of the dual-chamber spray device 800 in accordance with an example.

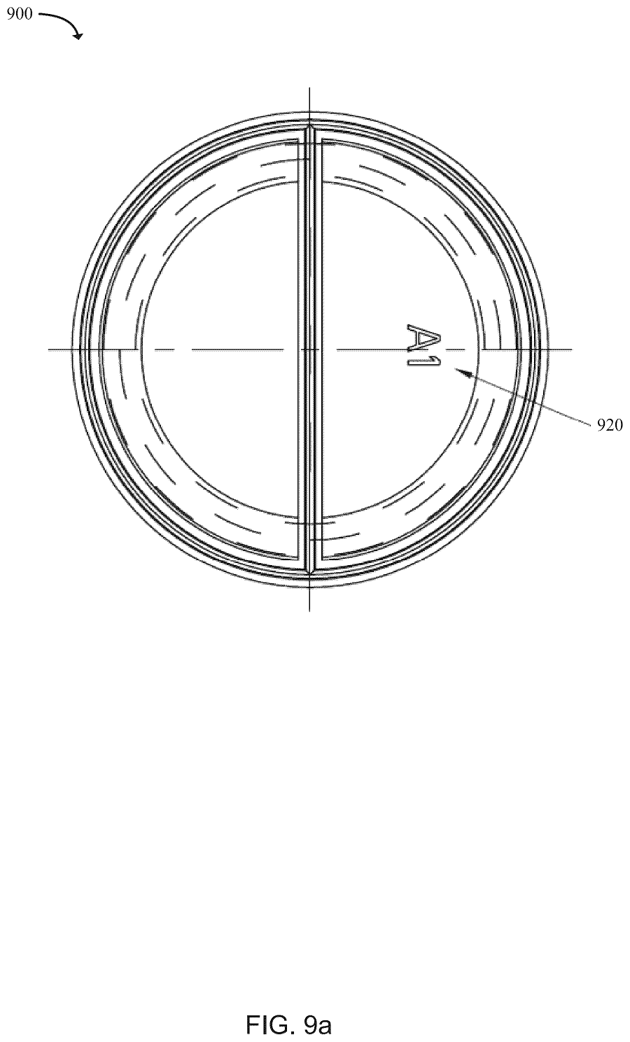

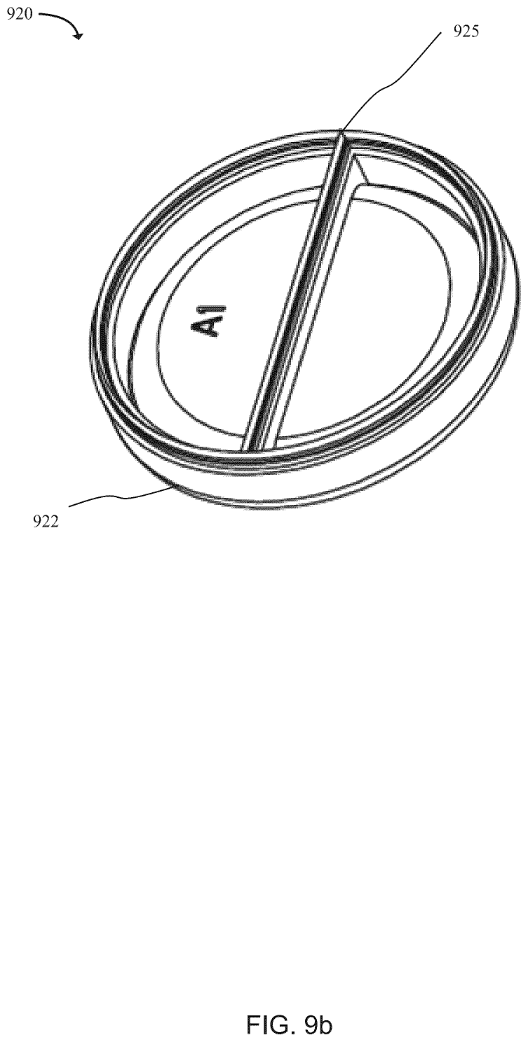

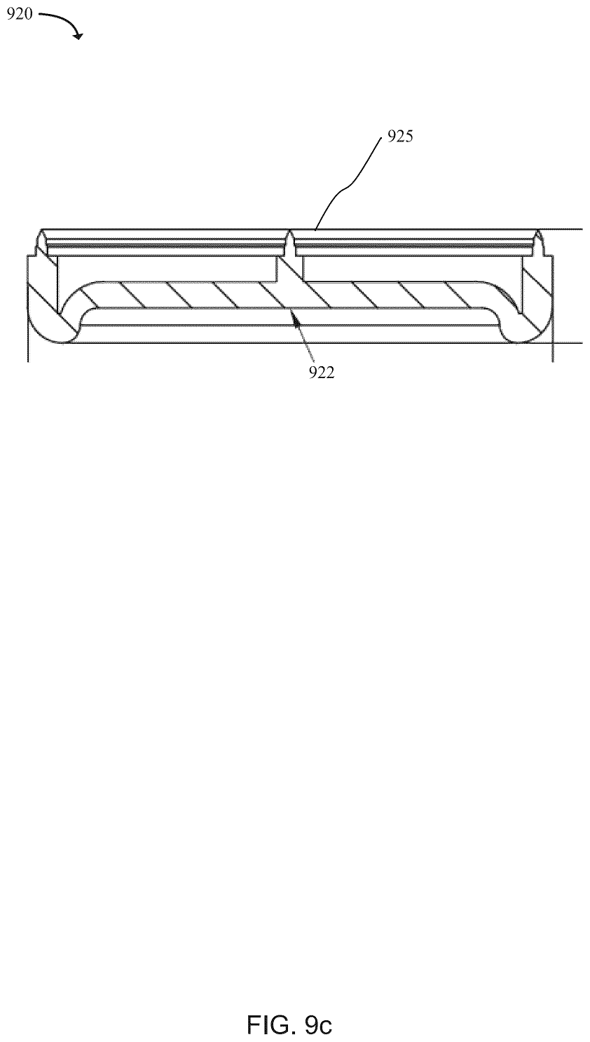

In another example, FIG. 9a is a top view of a gasket 920 of a dual-chamber spray device 900 in accordance with an example. FIG. 9b is a perspective view of a gasket 920 of the dual-chamber spray device in accordance with an example. The gasket 920 can include a tongue 922 that can connect to a liquid container. The gasket can include a divider 925 that can prevent mixing of a first liquid and a second liquid before the first liquid and the second liquid enter the first mixing space. FIG. 9c is a side cross-sectional view of a gasket 920 of a dual-chamber spray device in accordance with an example. The gasket 920 can include a tongue 922 and a divider 925.

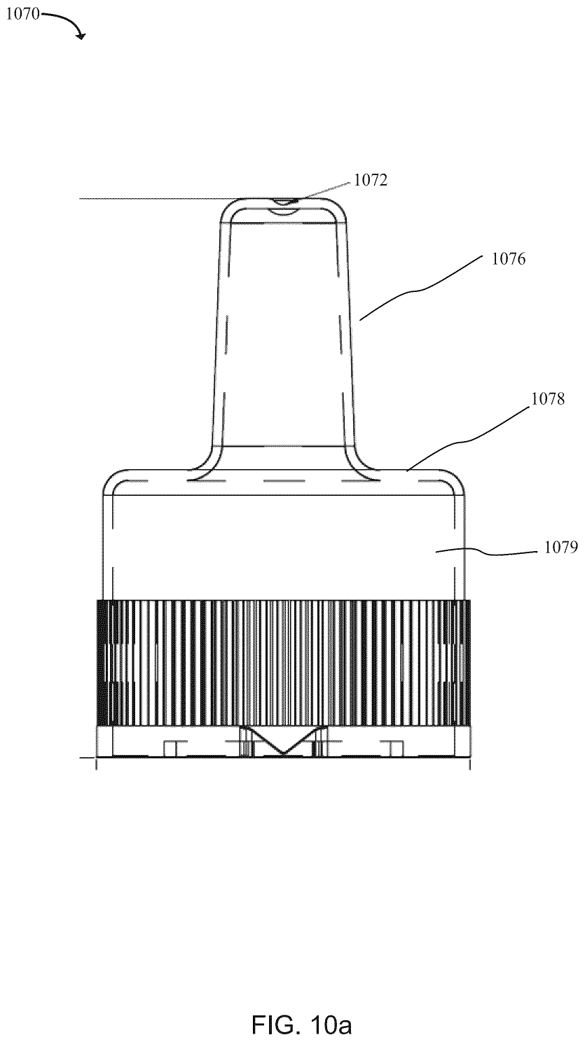

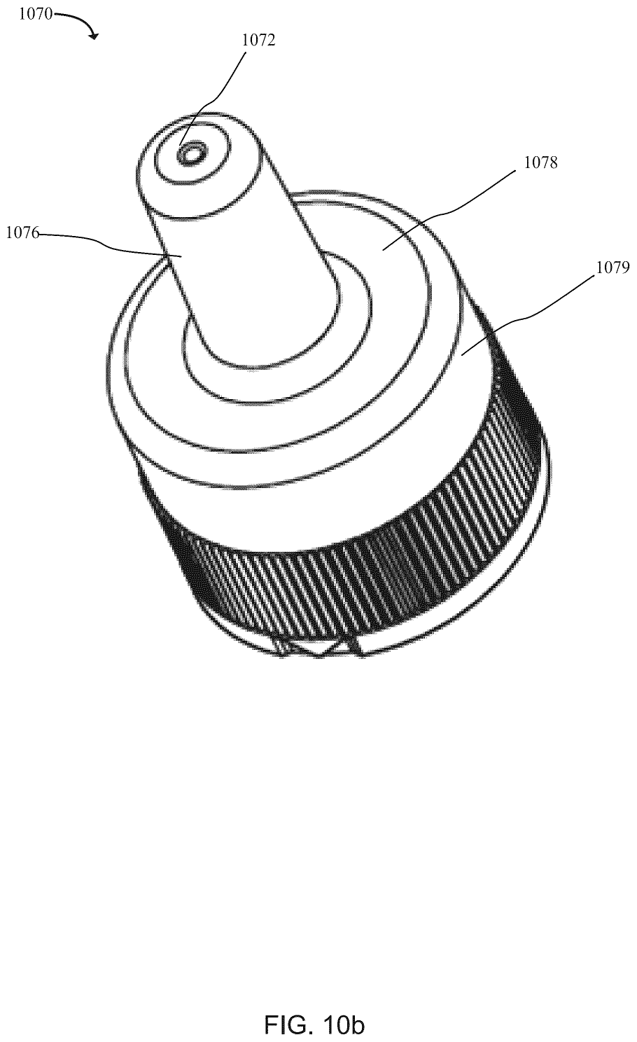

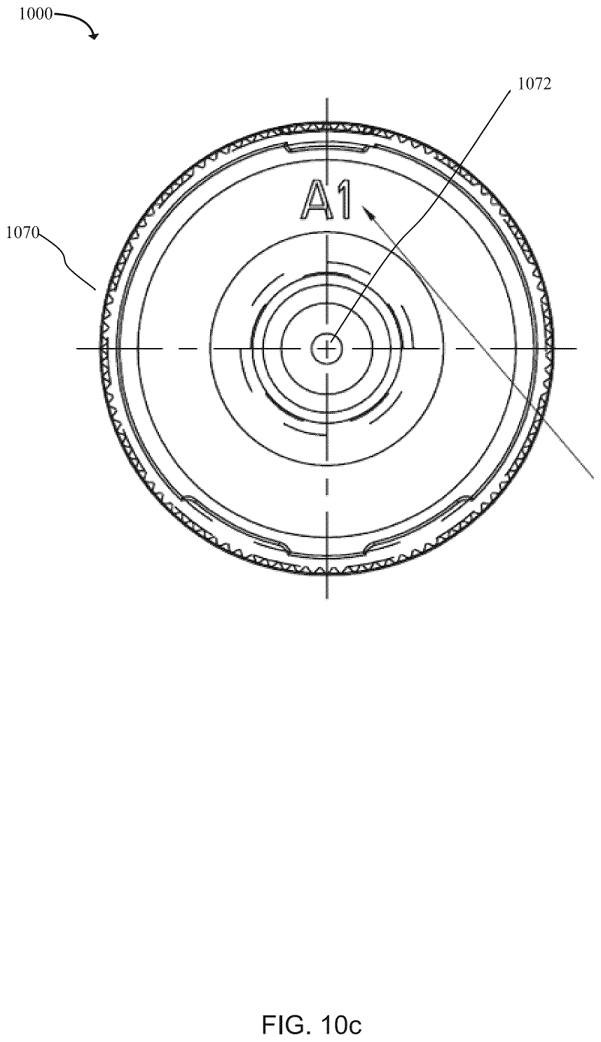

In one example, FIG. 10a is a side view of a top housing 1070 of a dual-chamber spray device 1000 in accordance with an example. The top housing can include a nozzle opening 1072, a third mixing space at a tip of the top housing, a second mixing space, a post cover 1076, a post collar 1078, and a sidewall 1079. FIG. 10b is a perspective view of a top housing 1070 of a dual-chamber spray device in accordance with an example. FIG. 10c is a top view of a top housing 1070 of a dual-chamber spray device in accordance with an example.

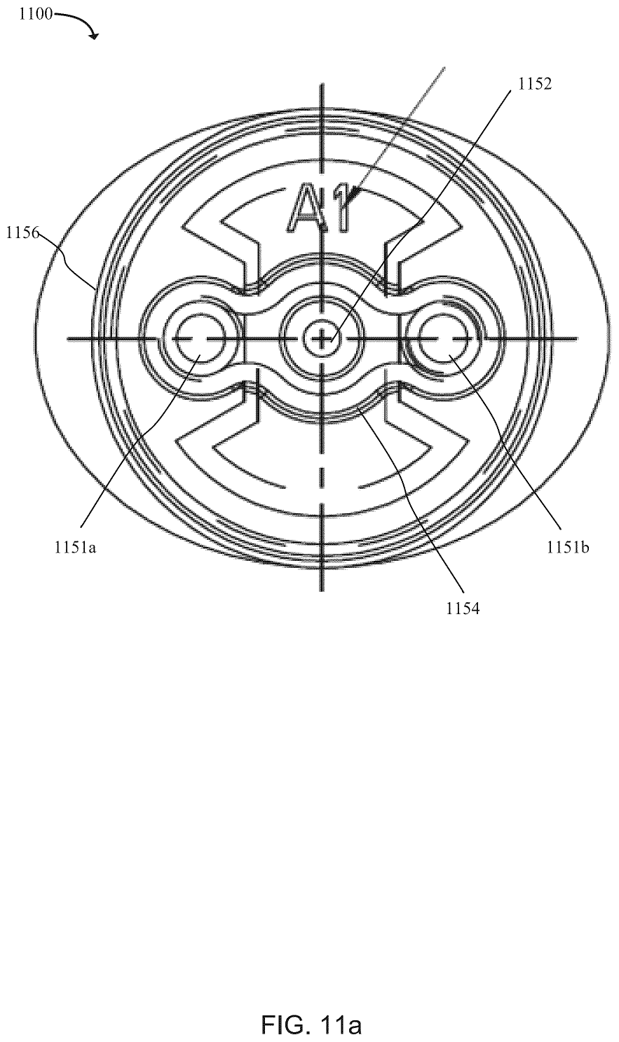

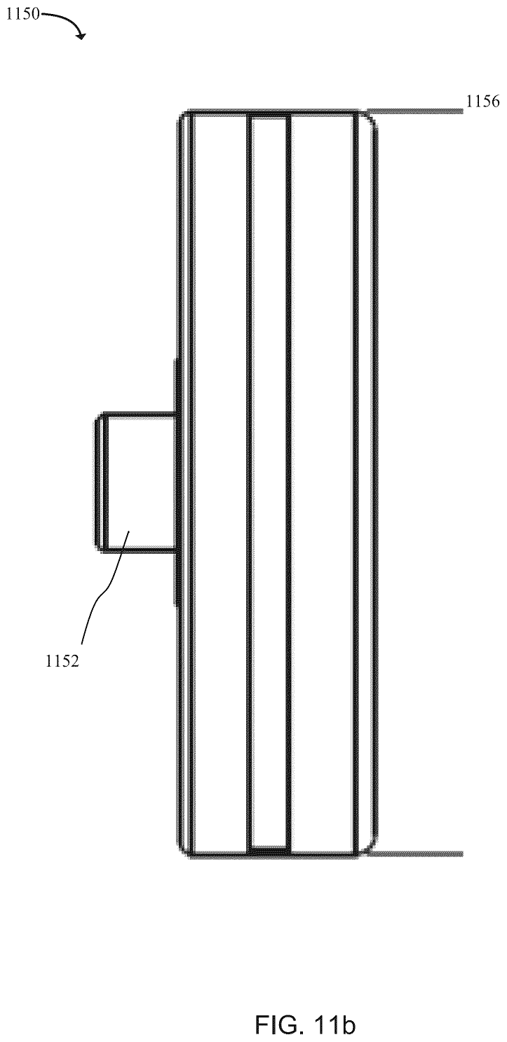

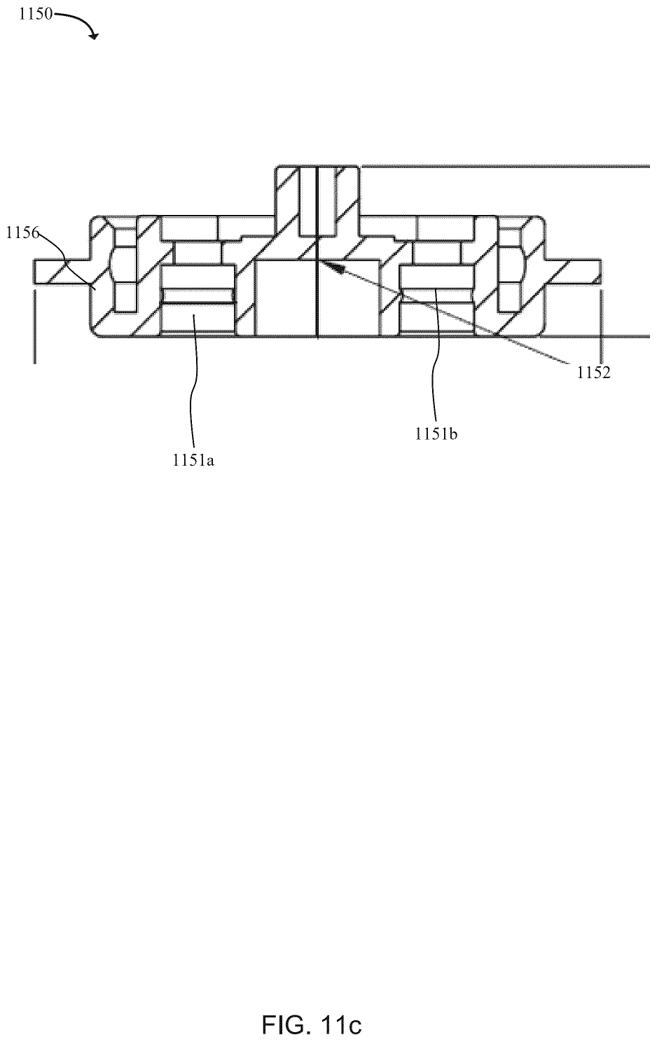

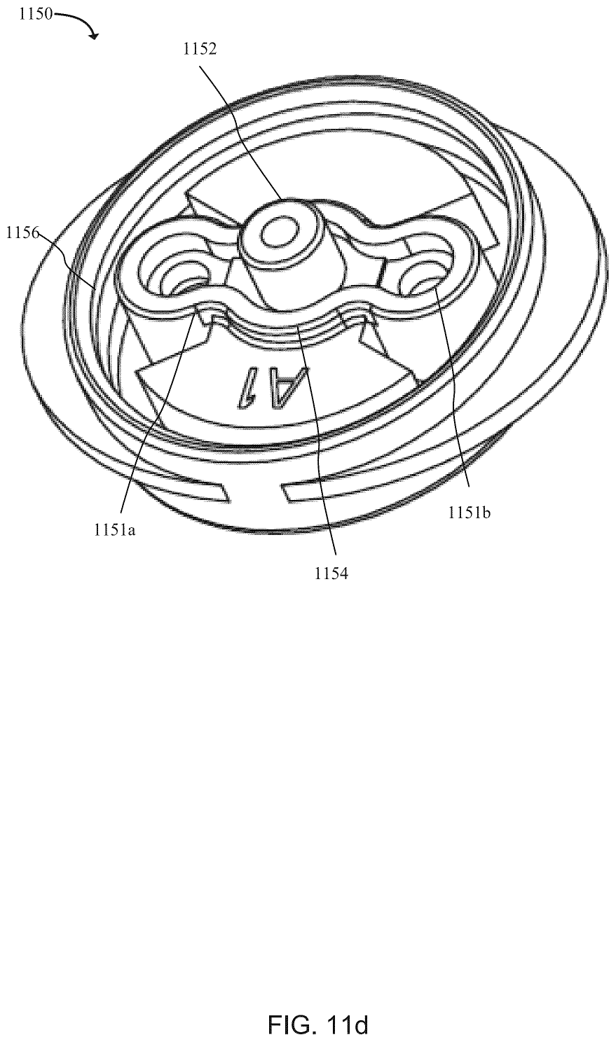

In another example, FIG. 11a is top view of a connecting plate 1150 of a dual-chamber spray device 1100 in accordance with an example. The connecting plate can include a first fluid inlet 1151a, a second fluid inlet 1151b, a central abutment 1152, an inner perimeter wall 1154, and an outer perimeter wall 1156. FIG. 11b is side view of the connecting plate 1150 of a dual-chamber spray device in accordance with an example. The connecting plate 1150 can include a central abutment 1152 and an outer perimeter wall 1156. FIG. 11c is side cross-sectional view of a connecting plate 1150 of a dual-chamber spray device in accordance with an example. The connecting plate can include a first fluid inlet 1151a, a second fluid inlet 1151b, a central abutment 1152, and an outer perimeter wall 1156. FIG. 11d is perspective view of a connecting plate 1150 of a dual-chamber spray device in accordance with an example. The connecting plate can include the central abutment 1152, the first fluid inlet 1151a, the second fluid inlet 1151b, and inner perimeter wall 1154, and the outer perimeter wall 1156.

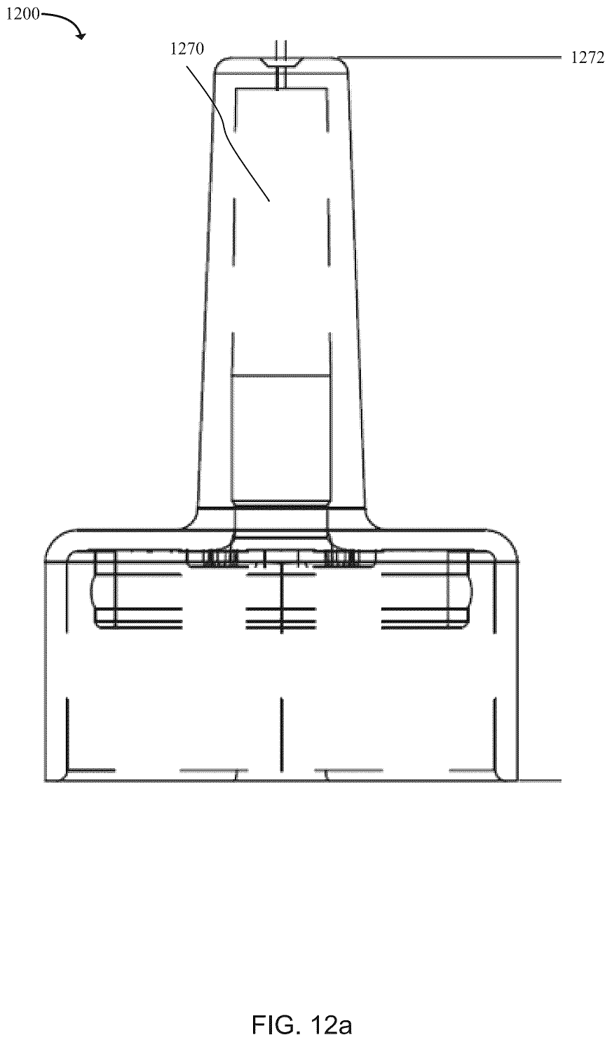

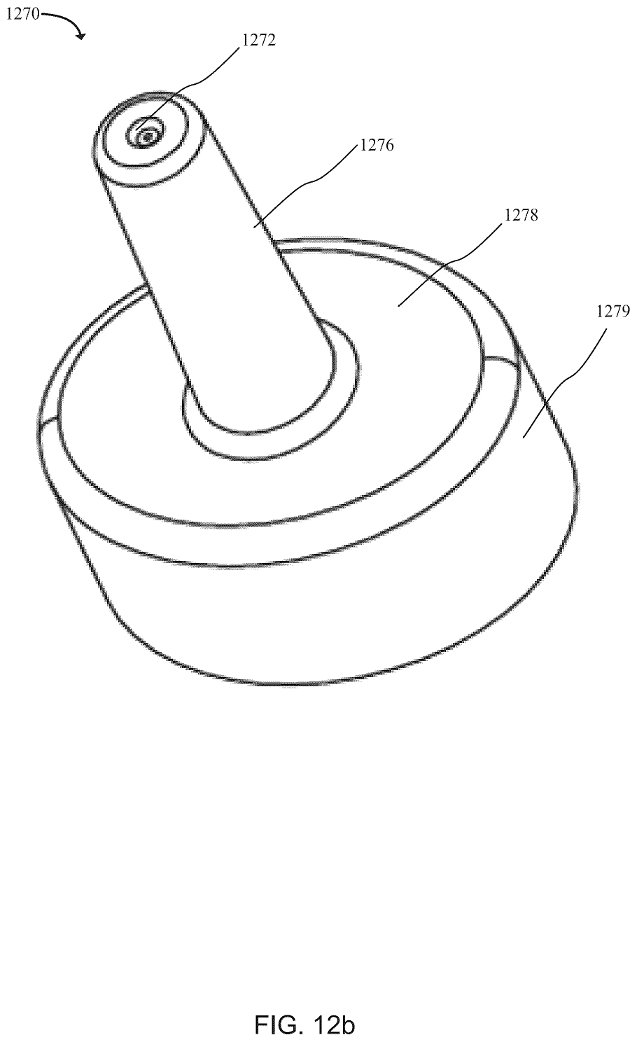

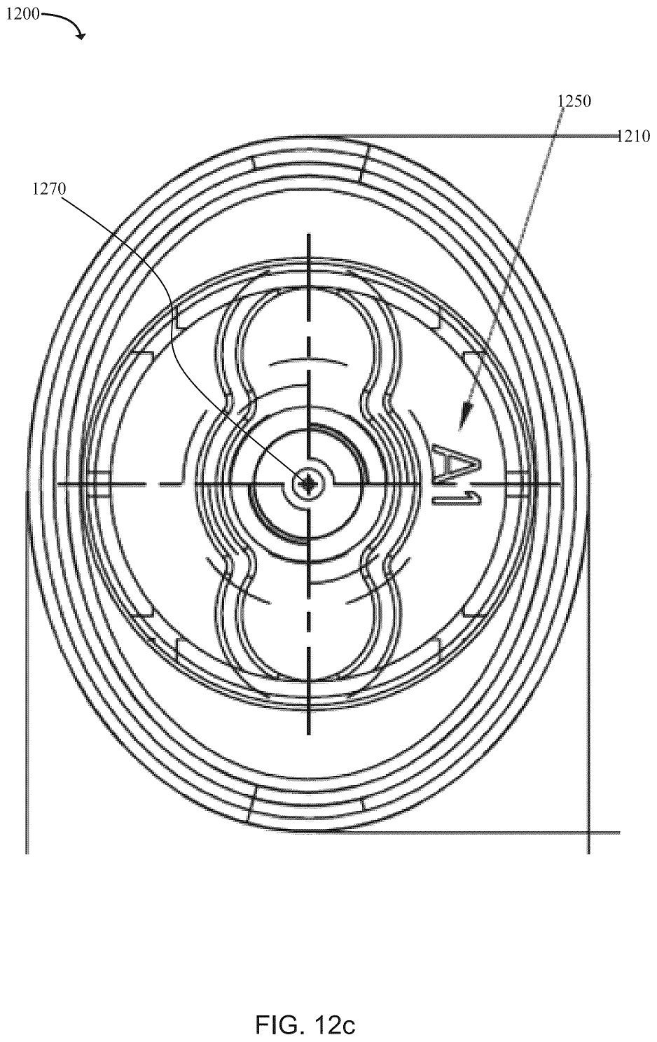

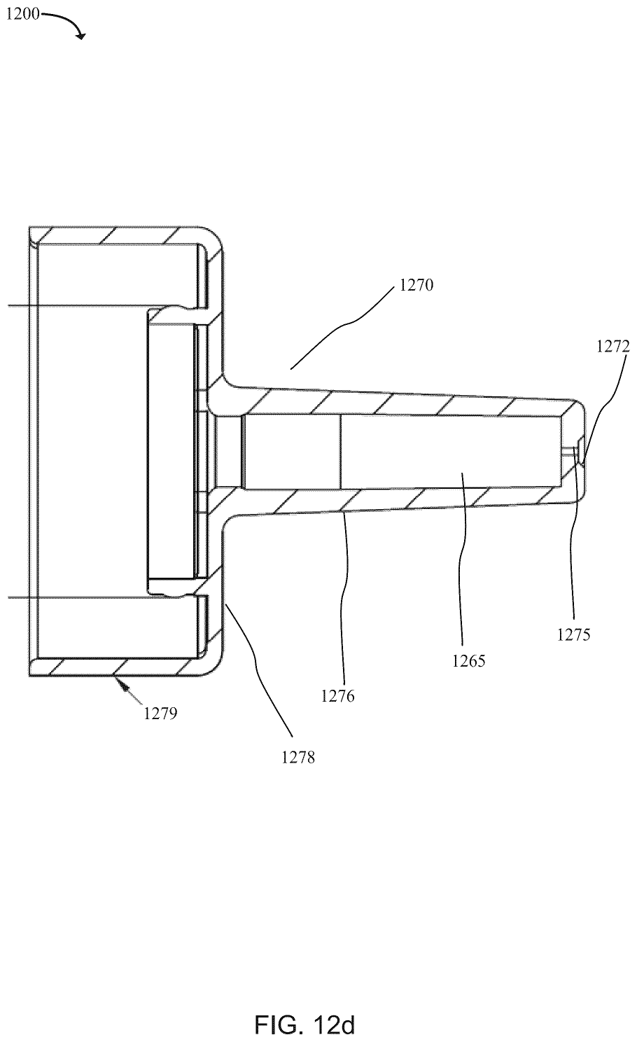

In another example, FIG. 12a illustrates a side view of a top housing 1270 of a dual-chamber spray device 1200 in accordance with an example. The top housing 1270 can include a nozzle opening 1272. FIG. 12b is perspective view of a top housing 1270 of a dual-chamber spray device that can include a nozzle opening 1272, a third mixing space at a tip of the top housing 1270, a second mixing space, a post cover 1276, a post collar 1278, and a sidewall 1279. FIG. 12c is top view of a top housing 1270 of a dual-chamber spray device 1200 in accordance with an example. The connecting plate 1250 can abut the top housing 1270 and the connecting plate can connect to the liquid container 1210. FIG. 12d is side cross-sectional view of a top housing 1270 of a dual-chamber spray device in accordance with an example. The top housing can include a nozzle opening 1272, a third mixing space 1275 at a tip of the top housing 1270, a second mixing space 1265, a post cover 1276, a post collar 1278, and a sidewall 1279.

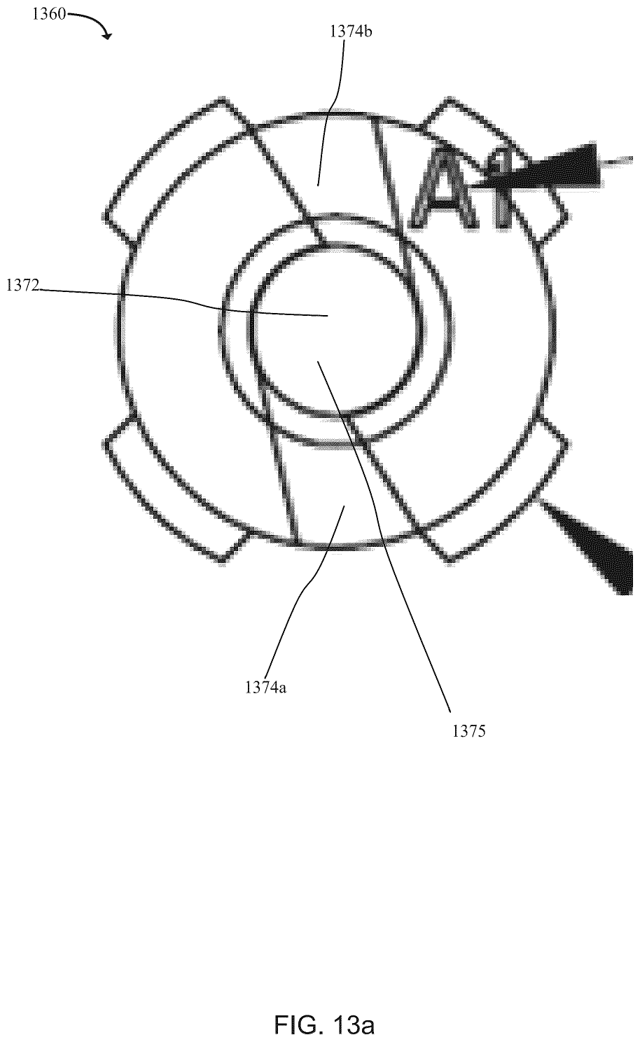

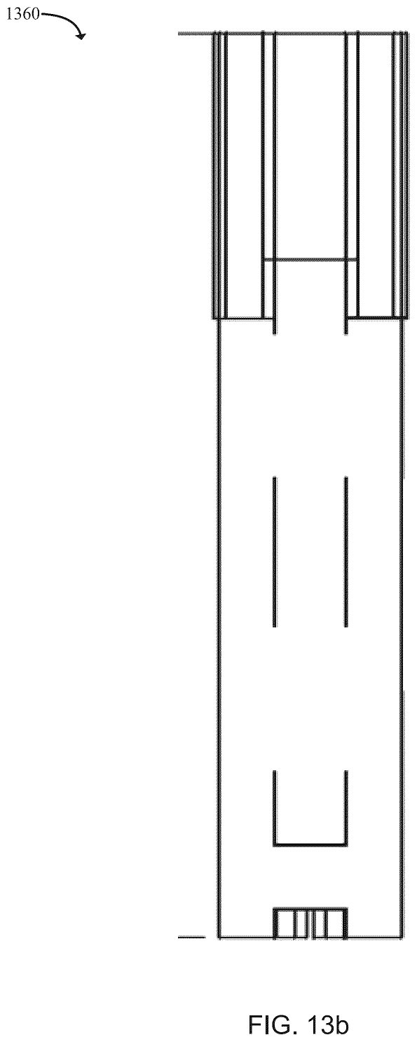

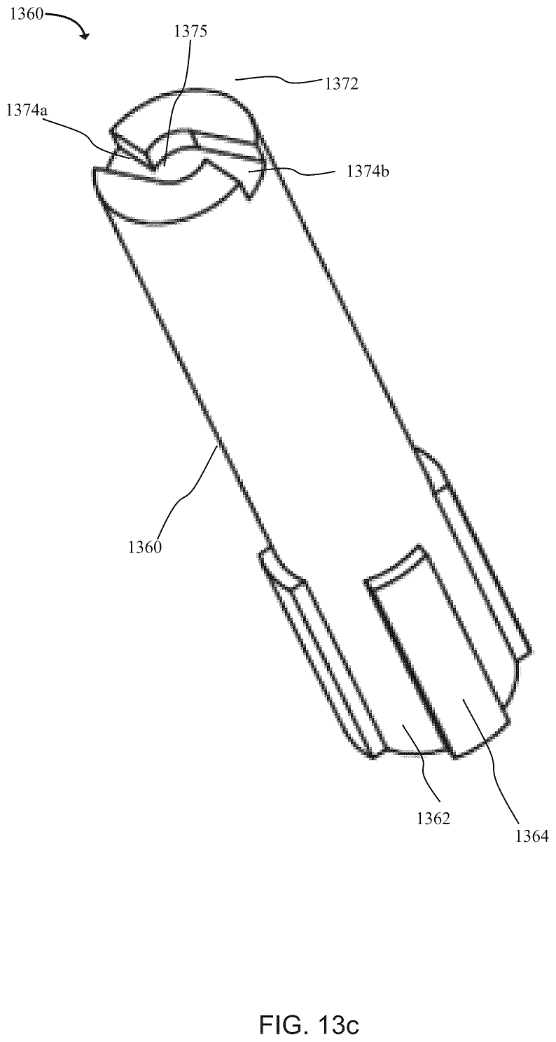

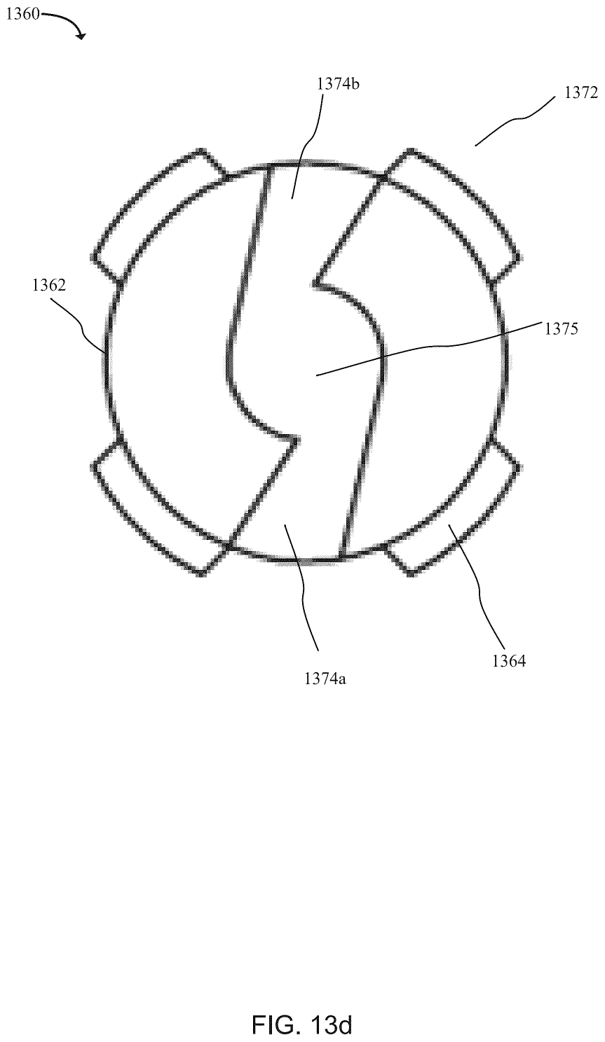

In another example, FIG. 13a is a top view of a post 1360 of a dual-chamber spray device in accordance with an example. The top of the post 1360 can include a first nozzle inlet 1374a, a second nozzle inlet 1374b, and a nozzle mixing space 1375. A mixed solution from the second mixing chamber can enter the first nozzle inlet 1374a and the second nozzle inlet 1374b from opposite directions of flow. The mixed solution can mix in the nozzle mixing space 1375 before exiting the spray device through the nozzle opening 1372. FIG. 13b is a side view of a post 1360 of a dual-chamber spray device in accordance with an example. FIG. 13c is a perspective view of a post 1360 of a dual-chamber spray device in accordance with an example. The post 1360 can include nozzle grooves 1362 and channel-forming protrusions 1364. The top of the post 1360 can include the first nozzle inlet, the second nozzle inlet 1374b, a third mixing space 1375, and the nozzle opening 1372. FIG. 13d is a top view of a post 1360 of a dual-chamber spray device in accordance with an example, in which the post 1360 includes nozzle grooves 1362, channel-forming protrusions 1364, a first nozzle inlet 1374a, a second nozzle inlet 1374b, a nozzle mixing space 1375, and a nozzle opening 1372.

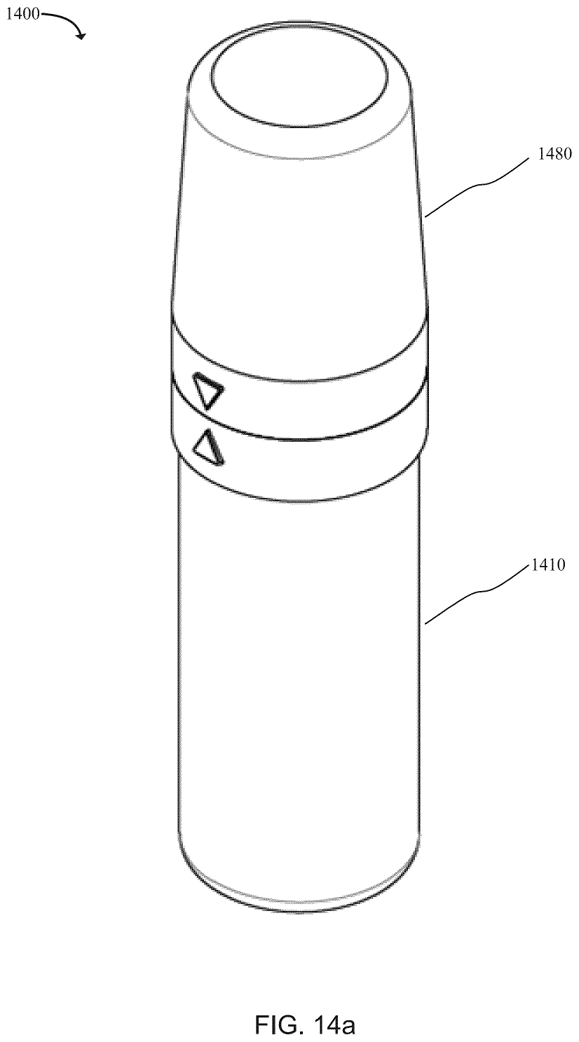

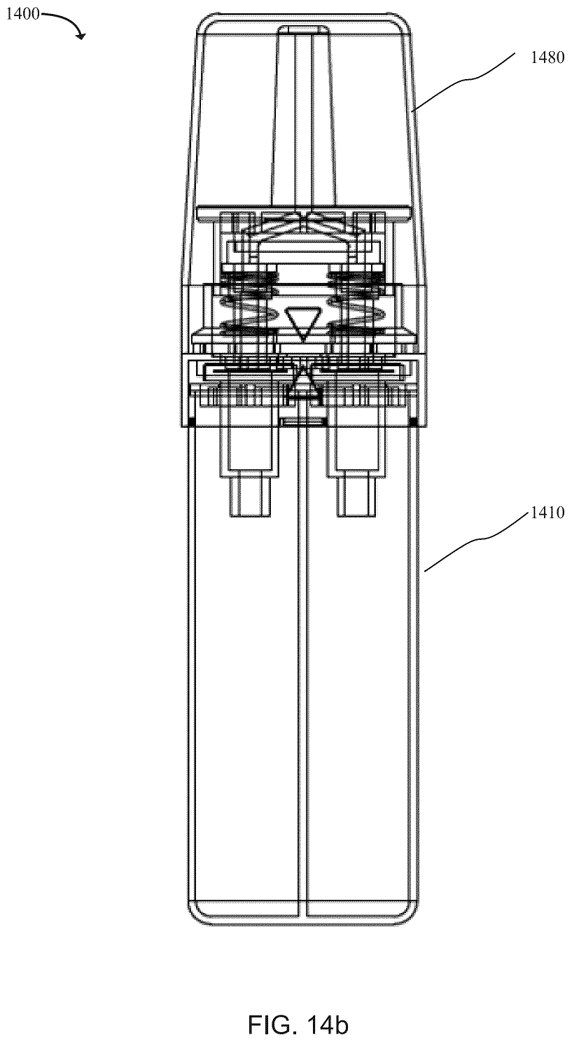

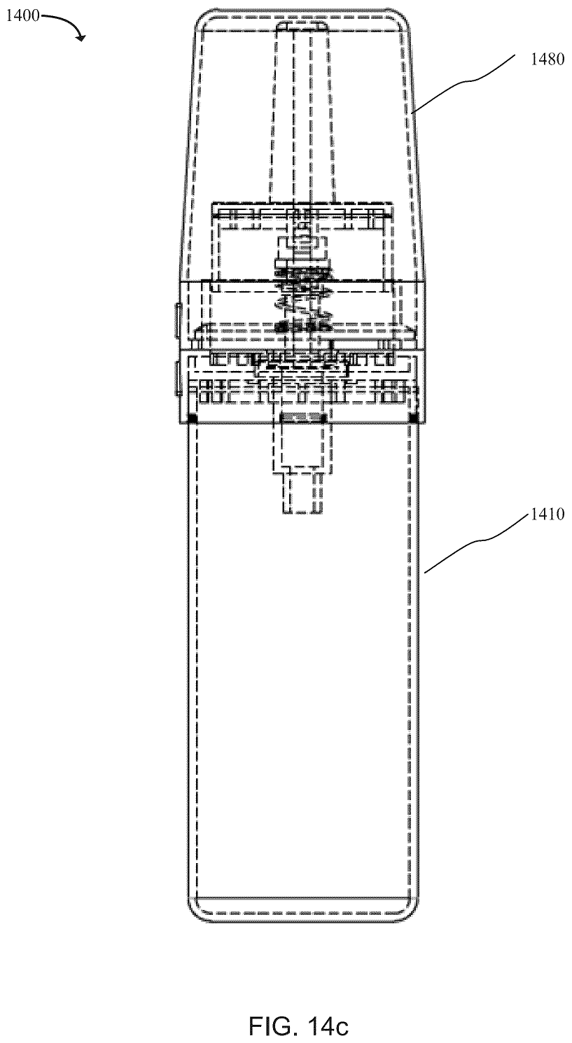

In another example, FIG. 14a is a perspective view of a dual-chamber spray device 1400 in accordance with an example. The dual-chamber spray device 1400 can include a liquid container 1410 and a safety cap 1480. FIG. 14b is a front view of a dual-chamber spray device 1400 that can include a liquid container 1410 and a safety cap 1480. FIG. 14c is a side view of a dual-chamber spray device 1400 that can include a liquid container 1410 and a safety cap 1480.

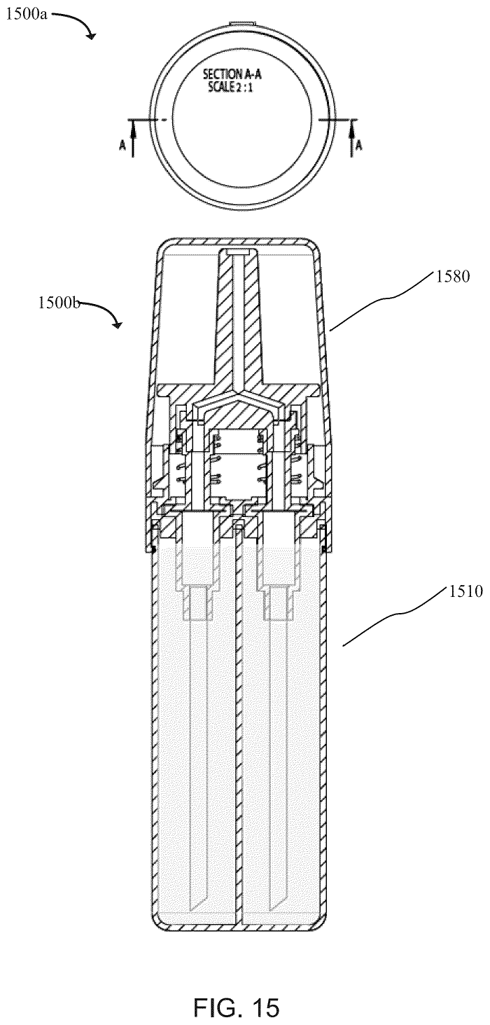

FIG. 15 is a front view of a cross-section 1500a of dual-chamber spray device 1500b in accordance with an example. The dual-chamber spray device 1500b can include a liquid container 1510 and a safety cap 1580.

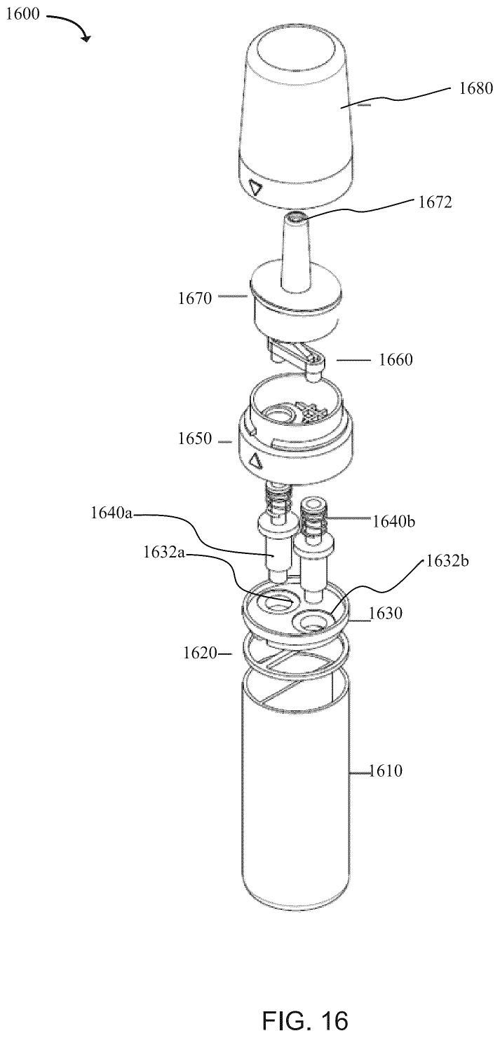

FIG. 16 is an exploded view of a dual-chamber spray device 1600 in accordance with an example. The dual-chamber spray device 1600 can include a liquid container 1610, a gasket 1620, a pump housing 1630 including a first pump fitting 1632a and a second pump fitting 1632b, a first pump 1640a, a second pump 1640b, a first connector 1650, a second connector 1660, a top housing 1670, a nozzle opening 1672, and a safety cap 1680.

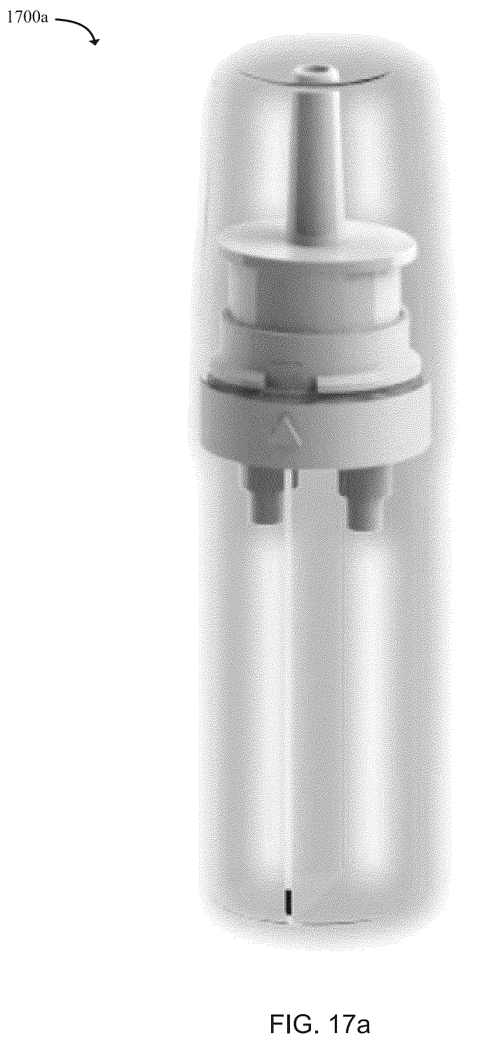

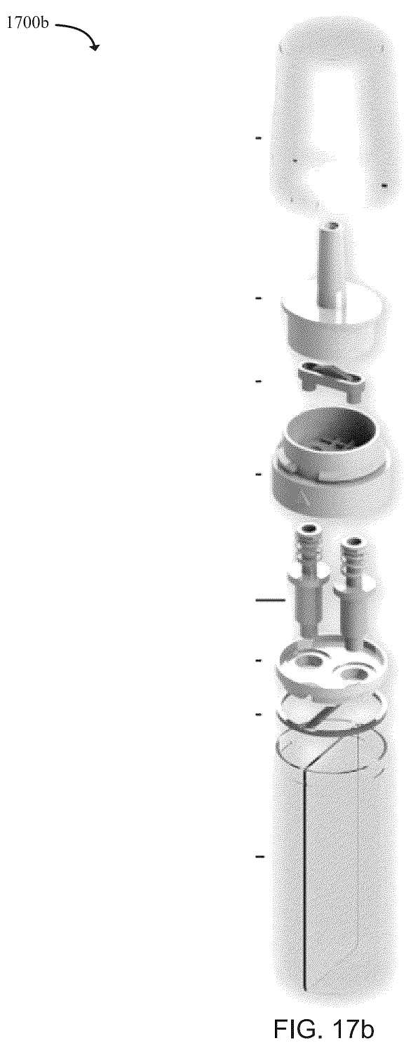

In another example, FIG. 17a illustrates a three-dimensional render of front view of a dual-chamber spray device 1700a. FIG. 17b is an exploded view of a dual-chamber spray device 1700b in accordance with an example.

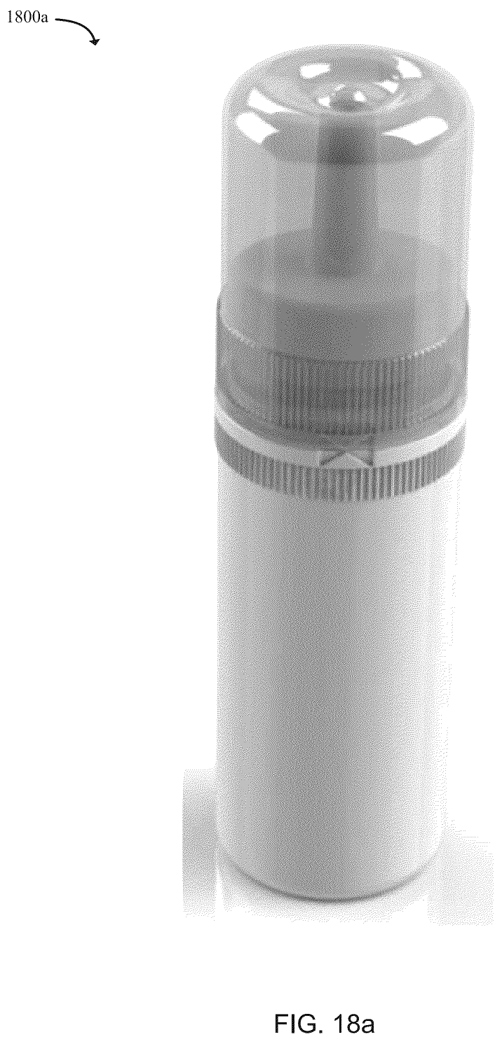

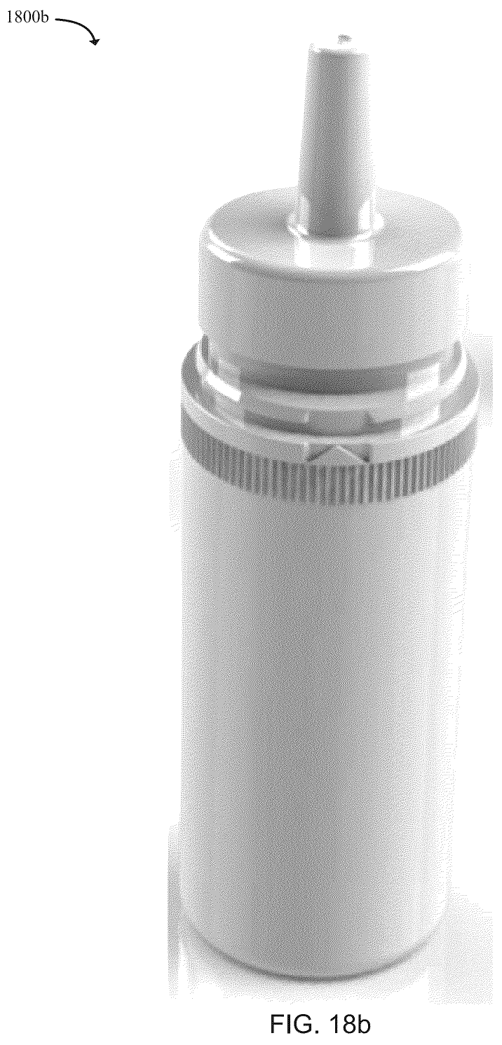

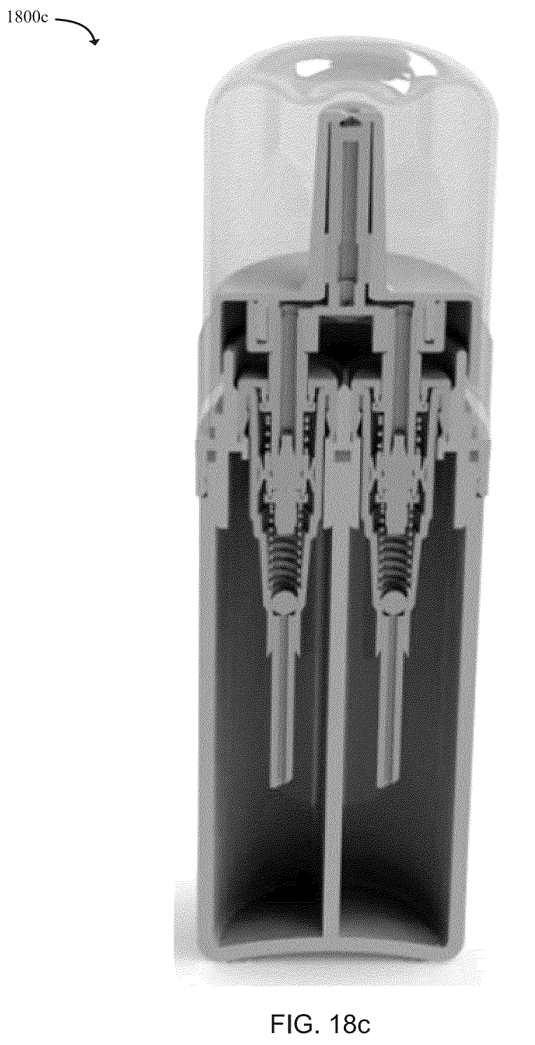

In another example, FIG. 18a is a three-dimensional render of a dual-chamber spray device 1800a in accordance with an example. FIG. 18b is a three-dimensional render of a dual-chamber spray device 1800b in accordance with an example. FIG. 18c is a three-dimensional render of a dual-chamber spray device 1800c in accordance with an example.

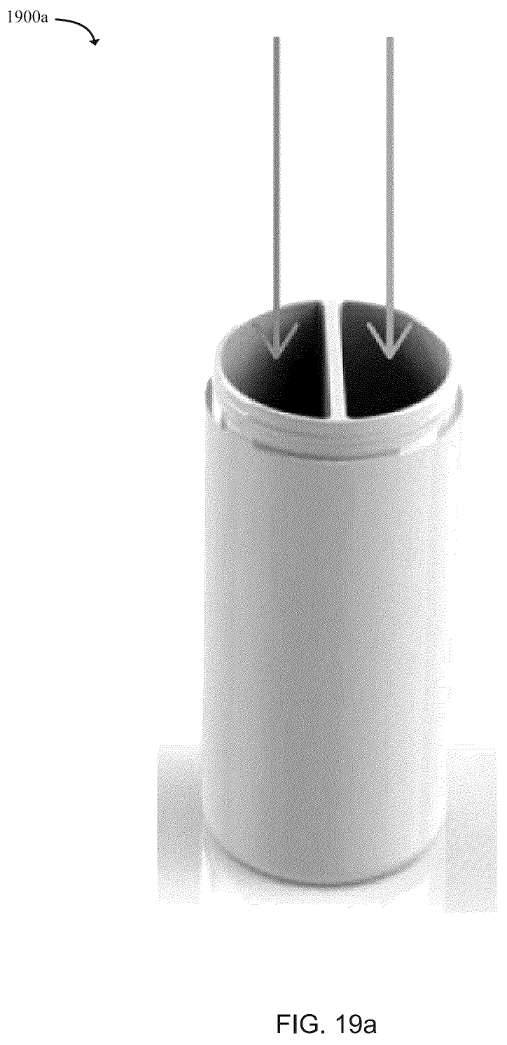

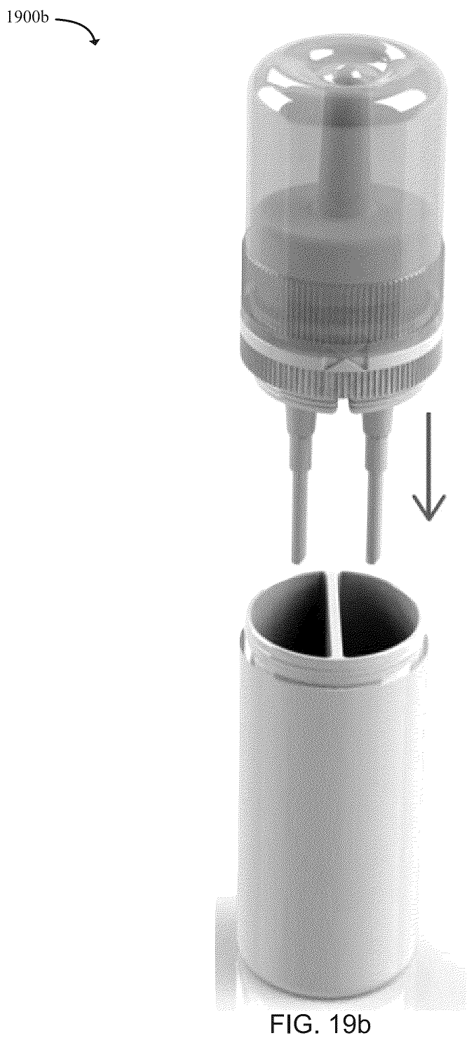

FIG. 19a is perspective view of a three-dimensional render of a dual-chamber spray device 1900a in accordance with an example. FIG. 19b is perspective view of a three-dimensional render of a dual-chamber spray device 1900b in accordance with an example.

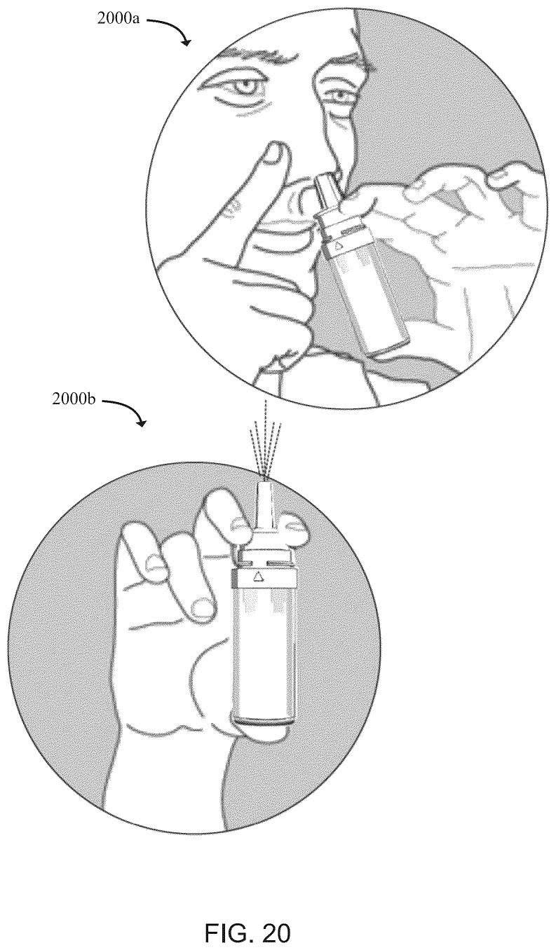

FIG. 20 illustrates a method of use 2000a and 200b of a dual-chamber spray device in accordance with an example.

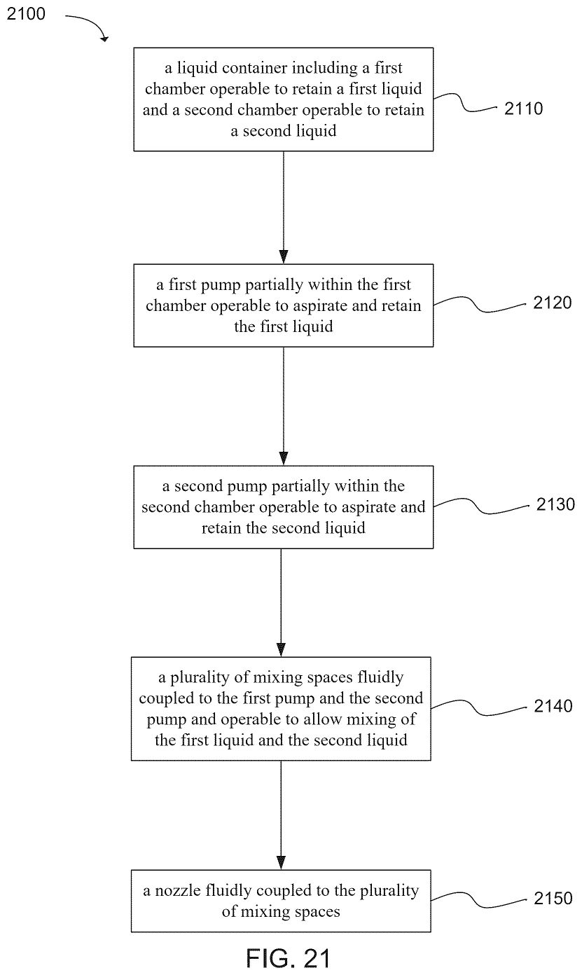

In one embodiment, as depicted in the flowchart in FIG. 21, a spray device 2100 can comprise a liquid container including a first chamber operable to retain a first liquid and a second chamber operable to retain a second liquid, as in block 2110. The spray device can further comprise a first pump partially within the first chamber operable to aspirate and retain the first liquid, as in block 2120. The spray device can further comprise a second pump partially within the second chamber operable to aspirate and retain the second liquid, as in block 2130. The spray device can further comprise a plurality of mixing spaces fluidly coupled to the first pump and the second pump and operable to allow mixing of the first liquid and the second liquid, as in block 2140. The spray device can further comprise a nozzle fluidly coupled to the plurality of mixing spaces, as in block 2150.

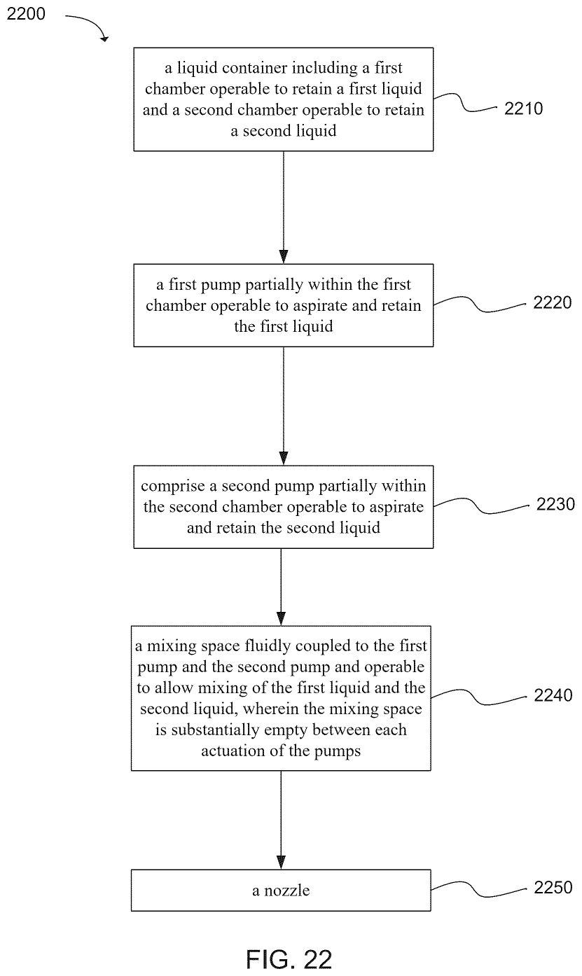

In one embodiment, as depicted in the flowchart in FIG. 22, a spray device 2200 can comprise a liquid container including a first chamber operable to retain a first liquid and a second chamber operable to retain a second liquid, as in block 2210. The spray device can further comprise a first pump partially within the first chamber operable to aspirate and retain the first liquid, as in block 2220. The spray device can further comprise a second pump partially within the second chamber operable to aspirate and retain the second liquid, as in block 2230. The spray device can further comprise a mixing space fluidly coupled to the first pump and the second pump and operable to allow mixing of the first liquid and the second liquid, wherein the mixing space is substantially empty between each actuation of the pumps, as in block 2240. The spray device can further comprise a nozzle, as in block 2250.

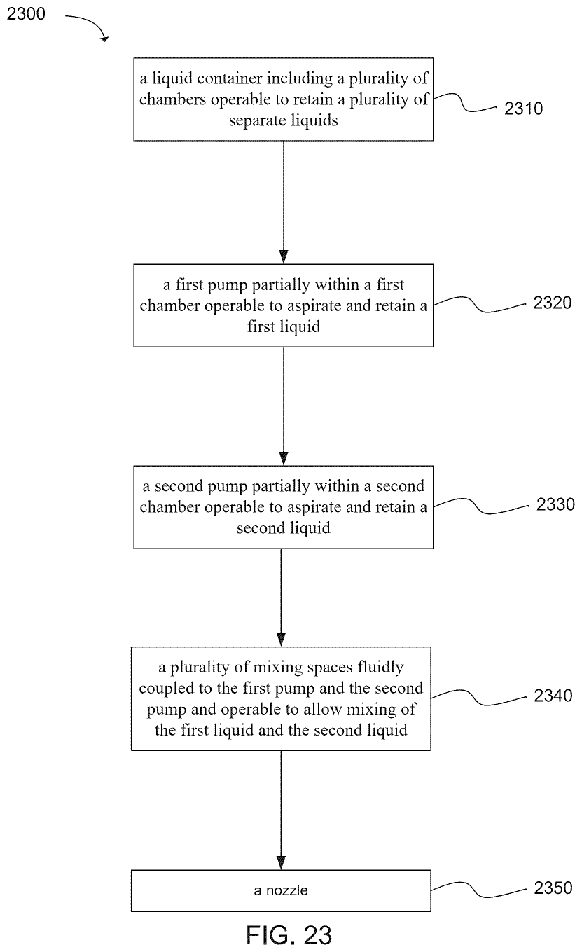

In one embodiment, as depicted in the flowchart in FIG. 23, a spray device 2300 can comprise a liquid container including a plurality of chambers operable to retain a plurality of separate liquids, as in block 2310. The spray device can further comprise a first pump partially within a first chamber operable to aspirate and retain a first liquid, as in block 2320. The spray device can further comprise a second pump partially within a second chamber operable to aspirate and retain a second liquid, as in block 2330. The spray device can further comprise a plurality of mixing spaces fluidly coupled to the first pump and the second pump and operable to allow mixing of the first liquid and the second liquid, as in block 2340. The spray device can further comprise a nozzle, as in block 2350.

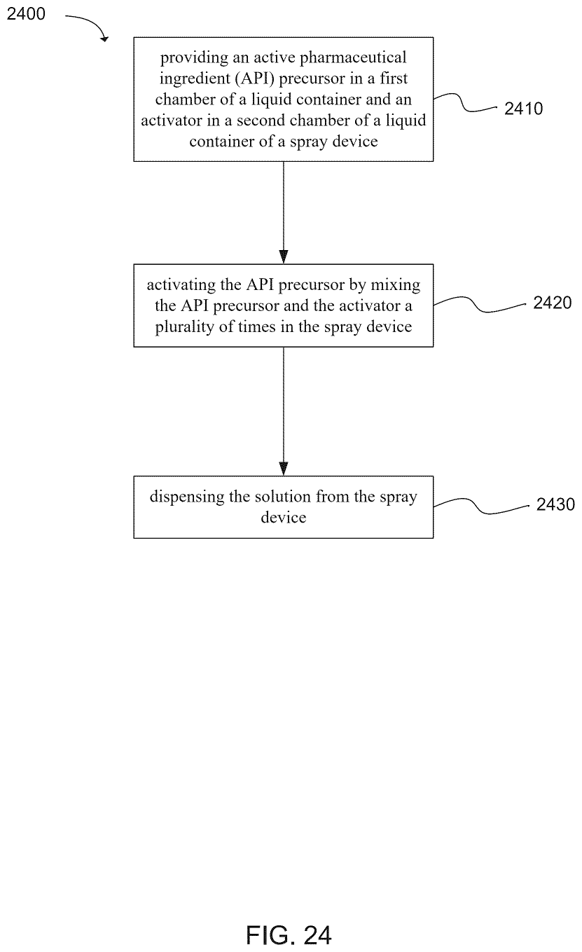

A method of dispensing a solution is also described herein. FIG. 24 depicts aspects of a method 2400 for dispensing a solution, as described in the flowchart. The method can comprise providing an active pharmaceutical ingredient (API) precursor in a first chamber of a liquid container and an activator in a second chamber of a liquid container of a spray device, as in block 2410. The method can comprise activating the API precursor by mixing the API precursor and the activator a plurality of times in the spray device, as in block 2420. The method can comprise dispensing the solution from the spray device, as in block 2430.

In addition, the method can include mixing the API precursor and the activator upon an act of dispensing the solution from the spray device. In one aspect, the method can include mixing the API precursor and the activator less than 50 milliseconds before dispensing the solution from the spray device. In another aspect, the method can include mixing the API precursor and the activator for a first time when the API precursor exits a first pump and the activator exits a second pump.

In another example, the method can include mixing the API precursor and the activator for a second time when a combination of the API precursor and the activator exits a first mixing space and enters a second mixing space of the spray device. In one aspect, the method can include mixing the API precursor and the activator for a third time when the combination of the API precursor and the activator enters a nozzle tip of the spray device.

In one example, the method can include mixing a ratio of a volume of the first chamber and a volume of the second chamber from about 1:10 to about 10:1. In one aspect, the method can include pressurizing the combination of the API precursor and the activator when dispensing the solution from the spray device. In another aspect, the method can include spraying the solution in an amount of from about 0.01 ml to about 5 ml per dispensing event. In another aspect, the method can include spraying the solution at an exit velocity from the spray device from about 6.5 m/s to about 19.5 m/s. In another example, the exit velocity can be from about 5 m/s to about 25 m/s. In another example, the exit velocity can be from about 13 m/s to about 19.5 m/s. In another aspect, the viscosity of the first liquid, or the second liquid, or a combination thereof can be from about 0.1 centipoise (cP) to about 10,000 cP.

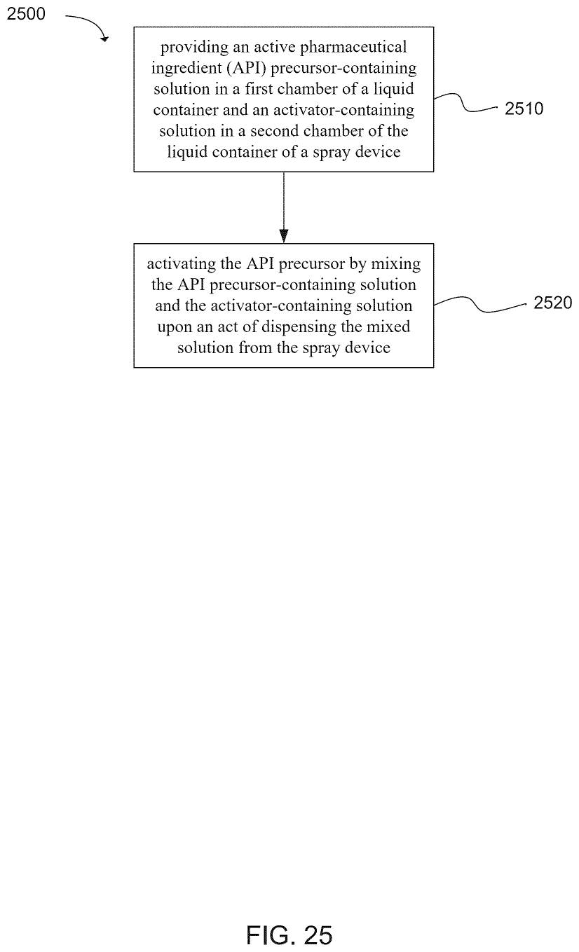

A method of activating a solution is also described herein. FIG. 25 depicts aspects of a method 2500 for activating a solution, as described in the flowchart. The method can comprise providing an active pharmaceutical ingredient (API) precursor-containing solution in a first chamber of a liquid container and an activator-containing solution in a second chamber of the liquid container of a spray device, as in block 2510. The method can comprise activating the API precursor by mixing the API precursor-containing solution and the activator-containing solution upon an act of dispensing the mixed solution from the spray device, as in block 2520.

In addition, the method can comprise mixing the API precursor-containing solution and the activator-containing solution less than 50 milliseconds before dispensing the mixed solution from the spray device. In one aspect, the method can comprise mixing the API precursor-containing solution and the activator-containing solution for a first time when the API precursor-containing solution exits a first pump and the activator-containing solution exits a second pump. In another aspect, the method can comprise mixing the API precursor-containing solution and the activator-containing solution for a second time when a combination of the API precursor-containing solution and the activator-containing solution exits a first mixing space and enters a second mixing space of the spray device. In another aspect, the method can comprise mixing the API precursor-containing solution and the activator-containing solution for a third time when the combination of the API precursor-containing solution and the activator-containing solution enters a nozzle tip of the spray device.

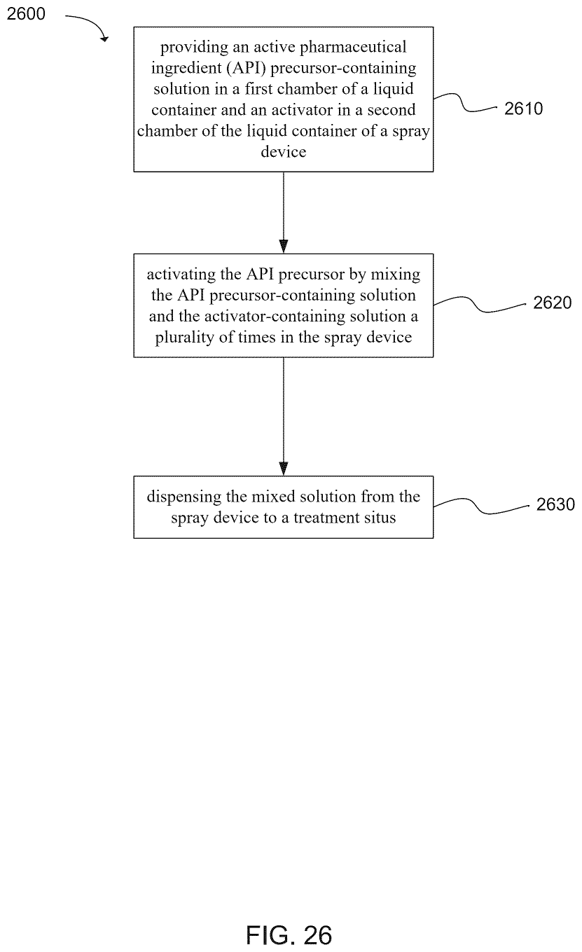

A method of administering a treatment is also described herein. FIG. 26 depicts aspects of a method 2600 for administering a treatment, as described in the flowchart. The method can comprise providing an active pharmaceutical ingredient (API) precursor-containing solution in a first chamber of a liquid container and an activator in a second chamber of the liquid container of a spray device, as in block 2610. The method can comprise activating the API precursor by mixing the API precursor-containing solution and the activator-containing solution a plurality of times in the spray device, as in block 2620. The method can comprise dispensing the mixed solution from the spray device to a treatment situs, as in block 2630.

In addition, the method can comprise mixing the API precursor-containing solution and the activator-containing solution for a first time when the API precursor-containing solution exits a first pump and the activator-containing solution exits a second pump. In one aspect, the method can comprise mixing the API precursor-containing solution and the activator-containing solution for a second time when a combination of the API precursor-containing solution and the activator-containing solution exits a first mixing space and enters a second mixing space of the spray device. In another aspect, the method can comprise mixing the API precursor-containing solution and the activator-containing solution for a third time when the combination of the API precursor-containing solution and the activator-containing solution enters a nozzle tip of the spray device.

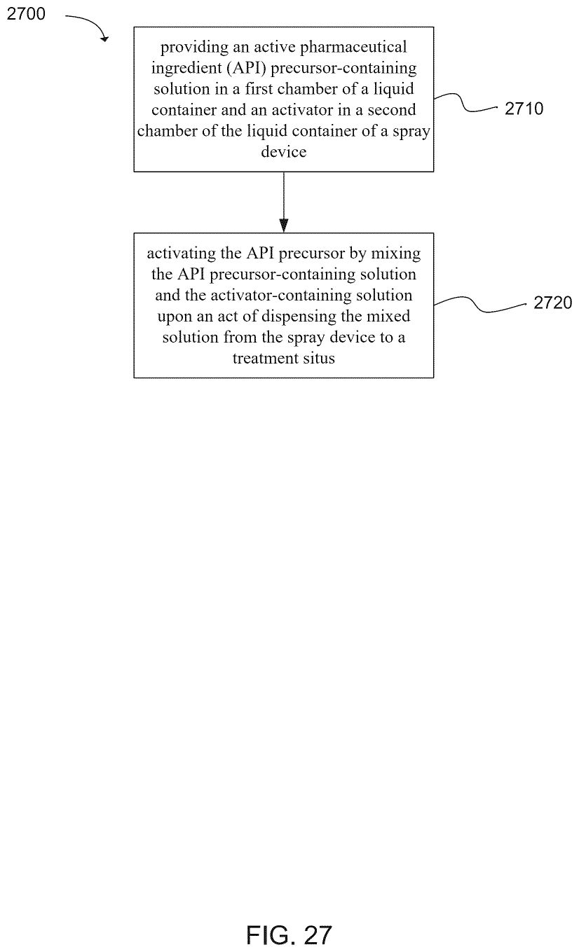

A method of administering a treatment is also described herein. FIG. 27 depicts aspects of a method 2700 for administering a treatment, as described in the flowchart. The method can comprise providing an active pharmaceutical ingredient (API) precursor-containing solution in a first chamber of a liquid container and an activator in a second chamber of the liquid container of a spray device, as in block 2710. The method can comprise activating the API precursor by mixing the API precursor-containing solution and the activator-containing solution upon an act of dispensing the mixed solution from the spray device to a treatment situs, as in block 2720.

In addition, the method can comprise mixing the API precursor-containing solution and the activator-containing solution less than 50 milliseconds before dispensing the mixed solution from the spray device.

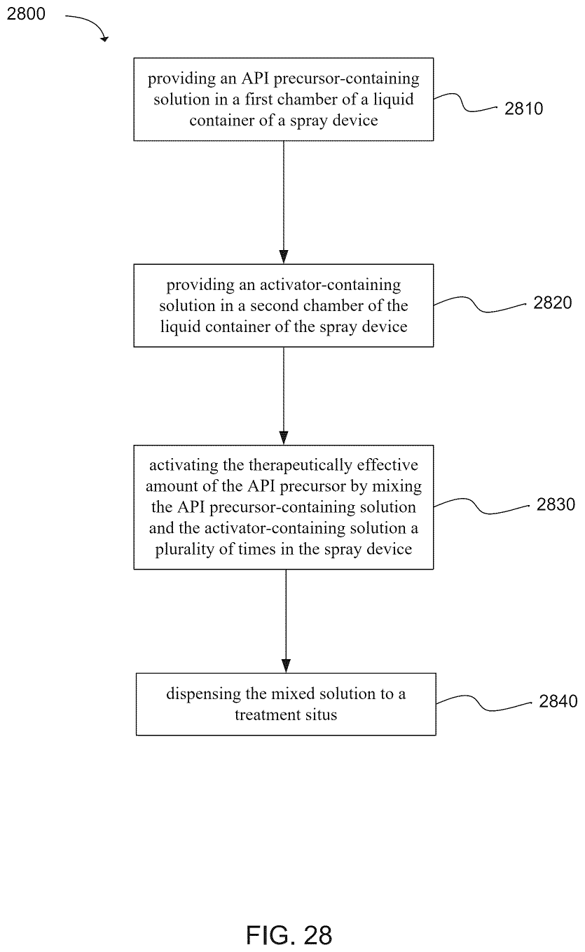

A method of providing a therapeutically effective amount of an unstable active pharmaceutical ingredient (API) is also described herein. FIG. 28 depicts aspects of a method 2800 for providing a therapeutically effective amount of an unstable active pharmaceutical ingredient (API), as described in the flowchart. The method can comprise providing an API precursor-containing solution in a first chamber of a liquid container of a spray device, as in block 2810. The method can comprise providing an activator-containing solution in a second chamber of the liquid container of the spray device, as in block 2820. The method can comprise activating the therapeutically effective amount of the API precursor by mixing the API precursor-containing solution and the activator-containing solution a plurality of times in the spray device, as in block 2830. The method can comprise dispensing the mixed solution to a treatment situs, as in block 2840

In addition, the method can include activating the therapeutically effective amount of the API upon an act of dispensing the mixed solution from the spray device. In one example, the method can include activating the therapeutically effective amount of the API less than 50 milliseconds before dispensing the mixed solution from the spray device. In another example, the method can include activating the therapeutically effective amount of the API by mixing a ratio of a volume of the API precursor and a volume of the activator from about 1:10 to about 10:1. In one aspect, the method can include activating the therapeutically effective amount of the API by pressurizing the combination of the API precursor and the activator when dispensing the mixed solution from the spray device.

In addition, the method can include mixing the API precursor-containing solution and the activator-containing solution for a first time when the API precursor-containing solution exits a first pump and the activator-containing solution exits a second pump. In one aspect, the method can include mixing the API precursor-containing solution and the activator-containing solution for a second time when a combination of the API precursor-containing solution and the activator-containing solution exits a first mixing space and enters a second mixing space of the spray device. In another aspect, the method can include mixing the API precursor-containing solution and the activator-containing solution for a third time when the combination of the API precursor-containing solution and the activator-containing solution enters a nozzle tip of the spray device.

In another embodiment, a method of manufacturing a product suitable for administering nitric oxide releasing solution (NORS) after an extended storage period can comprise providing a nitric oxide (NO) donor-containing solution in a first chamber of a liquid container of a spray device, and providing an activator-containing solution in a second chamber of the liquid container of the spray device. The method can further comprise activating the NORS by mixing the NO donor-containing solution and the activator-containing solution upon an act of dispensing the mixed solution from the spray device.

In one aspect, the extended storage period can be greater than at least one of: 5 days, 15 days, 30 days, 45 days, 90 days, 120 days, 180 days, one year, two years, or five years. In another aspect, a potency of the activated NORS after an extended storage period relative to the activated NORS potency before the extended storage period can be greater than one or more of: 70%, 80%, 90%, 95%, 99%, or 99.9%. The potency can be defined as the concentration (EC₅₀) or dose (ED₅₀) of a drug required to produce 50% of that drug's maximal effect. For example, when the potency of solution administered from the spray device has an EC₅₀ of 25 micromolar (μM) before an extended period of 180 days and the potency of solution administered from the spray device has an EC₅₀ of 20 μM after an extended period, then the potency of the activated NORS after an extended storage period relative to the activated NORS potency before the extended storage period can be about 80%.

In one aspect, the NO donor-containing solution and the activator-containing solution can be mixed less than a selected period of time before dispensing the mixed solution from the spray device. The selected period of time can be less than one or more of 15 seconds, 5 seconds, 1 second, 100 milliseconds (ms), 50 ms, or 10 ms. In one aspect, the method can comprise mixing the NO donor-containing solution and the activator-containing solution a plurality of times in the spray device upon the act of dispensing the mixed solution from the spray device.

It is noted that no specific order is required in these methods unless required by the claims set forth herein, though generally in some embodiments, the method steps can be carried out sequentially.

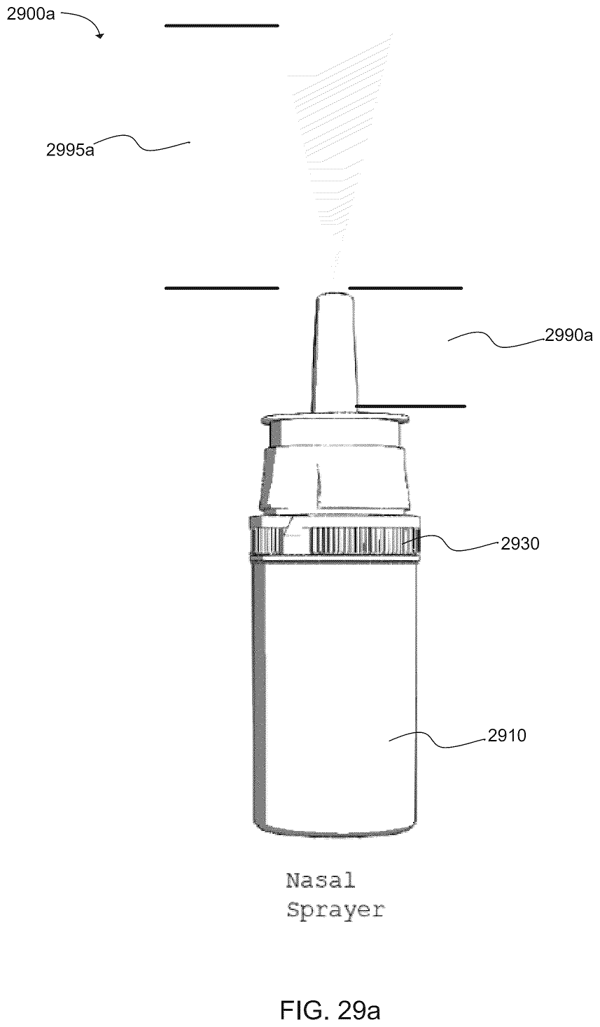

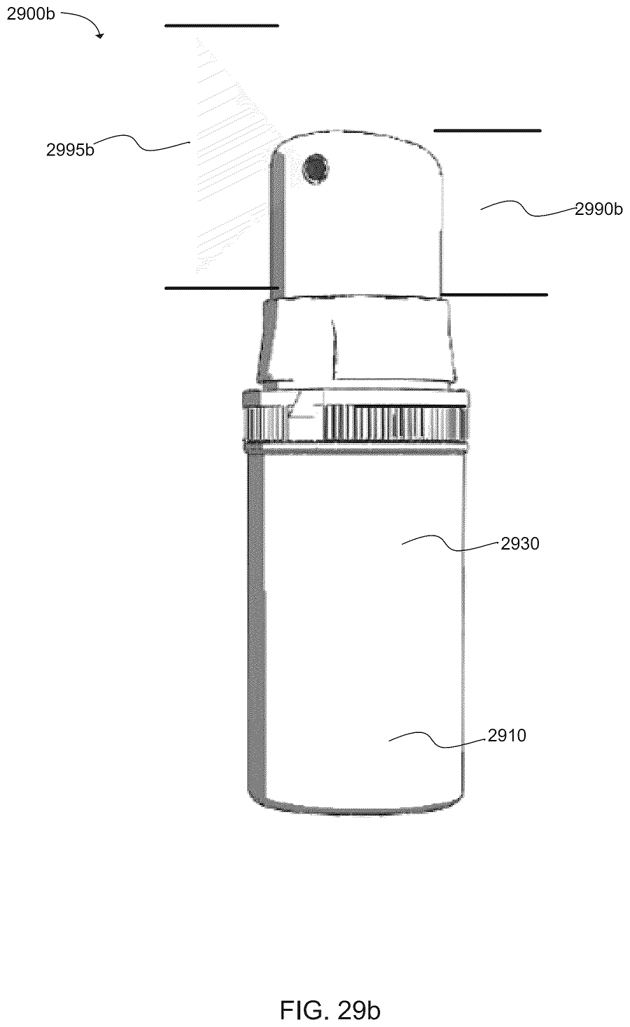

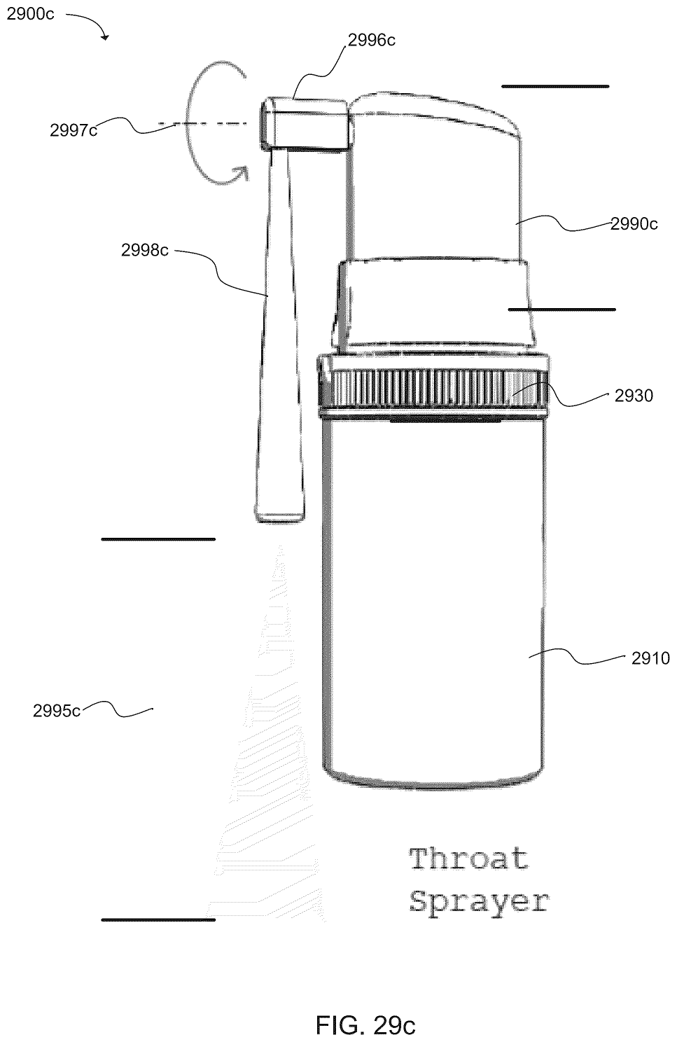

FIGS. 29a to 29c illustrate various shapes for a top housing of a spray device. The shape of the top housing can be configured to accommodate a selected indication or to administer the spray device to a selected treatment situs. For example, the spray device can have various shapes including a cone, a cube, a cylinder, a pyramid, a sphere, an ellipsoid, the like, and combinations thereof.

In one embodiment, as illustrated in FIG. 29a, the spray device can be a nasal spray device 2900a. The nasal spray device 2900a can include a liquid container 2910, a pump housing 2930, and other components discussed herein. The top housing of the nasal spray device 2900a can include a head 2990a that can be shaped as a cylinder and sized to be suitable for nasal administration to a subject. The nasal spray device 2900a can be configured to spray a spray pattern 2995a that can vary from one or more of a hollow cone, a full cone, a spiral cone, a solid stream, a mist, a fog, a flat fan, or combinations thereof.

In another embodiment, as illustrated in FIG. 29b, the spray device can be a 90° angle spray device 2900b. The spray device 2900b can include a liquid container 2910, a pump housing 2930, and other components discussed herein. The top housing of the spray device 2900b can include a head 2990b that can be shaped as a cylinder and sized to be suitable for topical administration to a subject. The head 2990b of the spray device 2900b can be configured to spray a spray pattern 2995b that can vary from one or more of a hollow cone, a full cone, a spiral cone, a solid stream, a mist, a fog, a flat fan, or combinations thereof.

In another embodiment, as illustrated in FIG. 29c, the spray device can be a throat spray device 2900c. The throat spray device 2900c can include a liquid container 2910, a pump housing 2930, and other components discussed herein. The top housing of the throat spray device 2900c can include a head 2990c that can include a mount 2996c, a pivot 2997c, and a tube 2998c. The tube 2998c can rotate around the pivot 2997c for ease of administration to a throat of a subject. The head 2990c of the throat spray device 2900c can be configured to spray a spray pattern 2995c that can vary from one or more of a hollow cone, a full cone, a spiral cone, a solid stream, a mist, a fog, a flat fan, or combinations thereof.

### EXAMPLE EMBODIMENTS

The following examples pertain to specific technology embodiments and point out specific features, elements, or steps that can be used or otherwise combined in achieving such embodiments.

In one example there is provided, a spray device (e.g. a nasal spray, throat spray, ear spray, medicament spray, or other spray device), comprising a liquid container including a first chamber operable to retain a first liquid and a second chamber operable to retain a second liquid; a first pump in fluid communication with the first chamber operable to aspirate and retain the first liquid; a second pump in fluid communication with the second chamber operable to aspirate and retain the second liquid; a plurality of mixing spaces fluidly coupled to the first pump and the second pump and operable to allow mixing of the first liquid and the second liquid; and a nozzle fluidly coupled to the plurality of mixing spaces.

In one example of a spray device, the plurality of mixing spaces is substantially empty between each actuation of the pumps.

In one example of a spray device, the plurality of mixing spaces includes: a first mixing space; and a second mixing space fluidly coupled between the first mixing space and the nozzle.

In one example of a spray device, the device further comprises a connecting plate including a first inlet, a second inlet, a central abutment, an inner perimeter wall defining at least a part of a first mixing space, and an outer perimeter wall.

In one example of a spray device, the first mixing space is fluidly coupled to a first outlet from the first pump and a second outlet from the second pump.

In one example of a spray device, the plurality of mixing spaces further includes a nozzle mixing space comprising a vortex tip of the nozzle.

In one example of a spray device, the device further comprises a connecting plate having a recess at least partially defining the first mixing space and openings fluidly coupling the first pump and the second pump to the first mixing space.

In one example of a spray device, the device further comprises: a post includinga plurality of nozzle grooves; and a plurality of channel-forming protrusions fluidly coupling a first mixing space to a second mixing space.

In one example of a spray device, the device further comprises a top housing including: a nozzle opening; and defining: a portion of a second mixing space; and a portion of a nozzle mixing space.

In one example of a spray device, the device further comprises a cap operable to be coupled to a top housing.

In one example of a spray device, the device further comprises a pump housing coupled between: the liquid container, and an outlet tube of the first pump and an outlet tube of the second pump; a first pump fitting configured to retain the first pump; a second pump fitting configured to retain the second pump; a base operable to connect to a connecting tongue of the liquid container; and a collar.

In one example of a spray device, the device further comprises a gasket coupled between the liquid container and a pump housing.

In one example of a spray device, the liquid container further comprises: a divider separating the first chamber and the second chamber; an outer housing; a bottom forming a bottom of the first chamber and the bottom of the second chamber; a connecting tongue operable to connect to a base of a pump housing.

In one example of a spray device, the first pump comprises: a first inlet tube in fluid communication with the first chamber, wherein the first inlet tube includes a first inlet, a first piston, a first spring, and a first ball valve; and a first outlet tube including a first outlet fluidly coupled to the first mixing space; and the second pump comprises: a second inlet tube in fluid communication with the second chamber, wherein the second inlet tube includes a second inlet, a second piston, a second spring, and a second ball valve; and a second outlet tube including a second outlet fluidly coupled to the first mixing space.

In one example of a spray device, a tip portion of the top housing is configured to spray at an angle ranging from about 0° to about 90° relative to a flow path out of the second mixing space; or the nozzle is configured to spray a predetermined pattern of liquid including one or more of a hollow cone, a full cone, a spiral cone, a solid stream, a mist, a fog, a flat fan, or combinations thereof. In one example of a spray device, a viscosity of the first liquid or the second liquid, or a combination thereof is from about 0.1 centipoise (cP) to about 10,000 cP. In one example of a spray device, a volume of the first chamber is from about 1 ml to about 100 ml; or a volume of the second chamber is from about 1 ml to about 100 ml.

In one example of a spray device, a ratio of a volume of the first chamber a volume of the second chamber is from about 1:10 to about 10:1.

In one example of a spray device, a volume of the first chamber and a volume of the second chamber is substantially equal.

In one example of a spray device, the spray device is operable to spray a liquid amount of from about 0.01 ml to about 5 ml upon each actuation of the first and second pumps.

In one example of a spray device, a ratio of a volume of the first mixing space to a volume of the second mixing space is from about 1:10 to about 10:1; or a ratio of a volume of the first mixing space to a volume of the third mixing space is from about 1000:1 to about 10:1; or a ratio of a volume of the second mixing space to a volume of the third mixing space is from about 1000:1 to about 10:1.

In one example of a spray device, the first pump is operable to aspirate and retain a liquid amount of from about 0.01 ml to about 5 ml; or the second pump is operable to aspirate and retain a liquid amount of from about 0.01 ml to about 5 ml.

In one example of a spray device, a ratio of a volume of: a capacity of the first pump or the second pump, and a volume of a first mixing space or a second mixing space is from about 100:1 to about 1:1; or a ratio of a volume of: a capacity of the first pump or the second pump, and a volume of a nozzle mixing space is from about 10000:1 to about 10:1.

In one example of a spray device, an exit velocity from the nozzle is from about 6.5 m/s to about 19.5 m/s.

In one example there is provided, a spray device comprising a liquid container including a first chamber operable to retain a first liquid and a second chamber operable to retain a second liquid; a first pump partially within the first chamber operable to aspirate and retain the first liquid; a second pump partially within the second chamber operable to aspirate and retain the second liquid; a mixing space fluidly coupled to the first pump and the second pump and operable to allow mixing of the first liquid and the second liquid, wherein the mixing space is substantially empty between each actuation of the pumps; and a nozzle.

In one example of a spray device, the device further comprises an additional mixing space fluidly coupled to the first pump and the second pump and operable to allow mixing of the first liquid and the second liquid.

In one example of a spray device, the device further comprises a nozzle mixing space comprising a vortex tip of the nozzle.

In one example of a spray device, the mixing space is fluidly coupled to a first outlet from the first pump and a second outlet from the second pump.

In one example there is provided, a spray device comprising: a liquid container including a plurality of chambers operable to retain a plurality of separate liquids; a first pump partially within a first chamber operable to aspirate and retain a first liquid; a second pump partially within a second chamber operable to aspirate and retain a second liquid; and a plurality of mixing spaces fluidly coupled to the first pump and the second pump and operable to allow mixing of the first liquid and the second liquid; and a nozzle. In one example of a spray device, the plurality of mixing spaces is substantially empty between each actuation of the pumps.

In one example of a spray device, the plurality of mixing spaces includes: a first mixing space; a second mixing space fluidly coupled between the first mixing space and the nozzle; and a nozzle mixing space fluidly coupled between the second mixing space and a nozzle opening.

In one example there is provided a method of dispensing a solution, comprising: providing an active pharmaceutical ingredient (API) precursor in a first chamber of a liquid container and an activator in a second chamber of a liquid container of a spray device; activating the API precursor by mixing the API precursor and the activator a plurality of times in the spray device; and dispensing the solution from the spray device. In one example of a method of dispensing a solution, the method further comprises mixing the API precursor and the activator upon an act of dispensing the solution from the spray device.

In one example of a method of dispensing a solution, the method further comprises mixing the API precursor and the activator less than 50 milliseconds before dispensing the solution from the spray device.

In one example of a method of dispensing a solution, the method further comprises mixing the API precursor and the activator for a first time when the API precursor exits a first pump and the activator exits a second pump.

In one example of a method of dispensing a solution, the method further comprises mixing the API precursor and the activator for a second time when a combination of the API precursor and the activator exits a first mixing space and enters a second mixing space of the spray device.

In one example of a method of dispensing a solution, the method further comprises mixing the API precursor and the activator for a third time when the combination of the API precursor and the activator enters a nozzle tip of the spray device. In one example of a method of dispensing a solution, the method further comprises mixing a ratio of a volume of the first chamber and a volume of the second chamber from about 1:10 to about 10:1.

In one example of a method of dispensing a solution, the method further comprises pressurizing the combination of the API precursor and the activator when dispensing the solution from the spray device.

In one example of a method of dispensing a solution, the method further comprises spraying the solution in an amount of from about 0.01 ml to about 5 ml per dispensing event.

In one example of a method of dispensing a solution, the method further comprises spraying the solution at an exit velocity from the spray device from about 6.5 m/s to about 19.5 m/s.

In one example of a method of dispensing a solution, a viscosity of the first liquid, or the second liquid, or a combination thereof is from about 0.1 centipoise (cP) to about 10,000 cP.

In one example there is provided a method of activating a solution, comprising providing an active pharmaceutical ingredient (API) precursor-containing solution in a first chamber of a liquid container and an activator-containing solution in a second chamber of the liquid container of a spray device; and activating the API precursor by mixing the API precursor-containing solution and the activator-containing solution upon an act of dispensing the mixed solution from the spray device.

In one example of a method of activating a solution, the method further comprises mixing the API precursor-containing solution and the activator-containing solution less than 50 milliseconds before dispensing the mixed solution from the spray device.

In one example of a method of activating a solution, the method further comprises mixing the API precursor-containing solution and the activator-containing solution for a first time when the API precursor-containing solution exits a first pump and the activator-containing solution exits a second pump.

In one example of a method of activating a solution, the method further comprises mixing the API precursor-containing solution and the activator-containing solution for a second time when a combination of the API precursor-containing solution and the activator-containing solution exits a first mixing space and enters a second mixing space of the spray device.

In one example of a method of activating a solution, the method further comprises mixing the API precursor-containing solution and the activator-containing solution for a third time when the combination of the API precursor-containing solution and the activator-containing solution enters a nozzle tip of the spray device.

In one example of a method of activating a solution, the method further comprises providing an active pharmaceutical ingredient (API) precursor-containing solution in a first chamber of a liquid container and an activator-containing solution in a second chamber of the liquid container of a spray device; activating the API precursor by mixing the API precursor-containing solution and the activator-containing solution a plurality of times in the spray device; and dispensing the mixed solution from the spray device to a treatment situs.

In one example of a method of activating a solution, the method further comprises mixing the API precursor-containing solution and the activator-containing solution for a first time when the API precursor-containing solution exits a first pump and the activator-containing solution exits a second pump.

In one example of a method of activating a solution, the method further comprises mixing the API precursor-containing solution and the activator-containing solution for a second time when a combination of the API precursor-containing solution and the activator-containing solution exits a first mixing space and enters a second mixing space of the spray device.

In one example of a method of activating a solution, the method further comprises mixing the API precursor-containing solution and the activator-containing solution for a third time when the combination of the API precursor-containing solution and the activator-containing solution enters a nozzle tip of the spray device.

In one example there is provided a method of administering a treatment, comprising: providing an active pharmaceutical ingredient (API) precursor-containing solution in a first chamber of a liquid container and an activator-containing solution in a second chamber of the liquid container of a spray device; activating the API precursor by mixing the API precursor-containing solution and the activator-containing solution upon an act of dispensing the mixed solution from the spray device to a treatment situs.

In one example of a method of administering a treatment, the method further comprises mixing the API precursor-containing solution and the activator-containing solution less than 50 milliseconds before dispensing the mixed solution from the spray device.

In one example there is provided a therapeutically effective amount of an unstable active pharmaceutical ingredient (API), comprising providing an API precursor-containing solution in a first chamber of a liquid container of a spray device providing an activator-containing solution in a second chamber of the liquid container of the spray device; and activating the therapeutically effective amount of the API precursor by mixing the API precursor-containing solution and the activator-containing solution a plurality of times in the spray device; and dispensing the mixed solution to a treatment situs.

In one example of a method of administering a treatment, the method further comprises activating the therapeutically effective amount of the API upon an act of dispensing the mixed solution from the spray device.

In one example of a method of administering a treatment, the method further comprises activating the therapeutically effective amount of the API less than 50 milliseconds before dispensing the mixed solution from the spray device. In one example of a method of administering a treatment, the method further comprises activating the therapeutically effective amount of the API by mixing a ratio of a volume of the API precursor and a volume of the activator from about 1:10 to about 10:1.

In one example of a method of administering a treatment, the method further comprises activating the therapeutically effective amount of the API by pressurizing the combination of the API precursor and the activator when dispensing the mixed solution from the spray device.

In one example of a method of administering a treatment, the method further comprises mixing the API precursor-containing solution and the activator-containing solution for a first time when the API precursor-containing solution exits a first pump and the activator-containing solution exits a second pump.

In one example of a method of administering a treatment, the method further comprises mixing the API precursor-containing solution and the activator-containing solution for a second time when a combination of the API precursor-containing solution and the activator-containing solution exits a first mixing space and enters a second mixing space of the spray device.

In one example of a method of administering a treatment, the method further comprises mixing the API precursor-containing solution and the activator-containing solution for a third time when the combination of the API precursor-containing solution and the activator-containing solution enters a nozzle tip of the spray device.

In one example there is provided a method of manufacturing a product suitable for administering nitric oxide releasing solution (NORS) after an extended storage period, comprising providing a nitric oxide (NO) donor-containing solution in a first chamber of a liquid container of a spray device; providing an activator-containing solution in a second chamber of the liquid container of the spray device; and activating the NORS by mixing the NO donor-containing solution and the activator-containing solution upon an act of dispensing the mixed solution from the spray device.

In one example of a method of manufacturing a product suitable for administering NORS after an extended storage period, the method further comprises mixing the NO donor-containing solution and the activator-containing solution a plurality of times in the spray device upon the act of dispensing the mixed solution from the spray device.

In one example of a method of manufacturing a product suitable for administering NORS after an extended storage period, the extended storage period is greater than at least one of: 5 days, 15 days, 30 days, 45 days, 90 days, 120 days, 180 days, one year, two years, or five years.

In one example of a method of manufacturing a product suitable for administering NORS after an extended storage period, an activated NORS potency after the extended storage period relative to the activated NORS potency before the extended storage period is greater than one or more of: 70%, 80%, 90%, 95%, 99%, or 99.9%.

In one example of a method of manufacturing a product suitable for administering NORS after an extended storage period, the NO donor-containing solution and the activator-containing solution is mixed less than a selected period of time before dispensing the mixed solution from the spray device.

In one example of a method of manufacturing a product suitable for administering NORS after an extended storage period, the selected period of time is less than one or more of 15 seconds, 5 seconds, 1 second, 100 milliseconds (ms), 50 ms, or 10 ms.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A spray device, comprising:
a liquid container including a first chamber operable to retain a first liquid and a second chamber operable to retain a second liquid;
a first pump in fluid communication with the first chamber operable to aspirate and retain the first liquid;
a second pump in fluid communication with the second chamber operable to aspirate and retain the second liquid;
a plurality of mixing spaces fluidly coupled to the first pump and the second pump and operable to allow mixing of the first liquid and the second liquid; and
a nozzle fluidly coupled to the plurality of mixing spaces.

2. The spray device of claim 1, wherein the plurality of mixing spaces is substantially empty between each actuation of the pumps.

3. The spray device of claim 1, wherein the plurality of mixing spaces includes:
a first mixing space; and
a second mixing space fluidly coupled between the first mixing space and the nozzle.

4. The spray device of claim 1, further comprising a connecting plate including a first inlet, a second inlet, a central abutment, an inner perimeter wall defining at least a part of a first mixing space, and an outer perimeter wall.

5. The spray device of claim 3, wherein the first mixing space is fluidly coupled to a first outlet from the first pump and a second outlet from the second pump.

6. The spray device of claim 3, wherein the plurality of mixing spaces further includes a nozzle mixing space comprising a vortex tip of the nozzle.

7. The spray device of claim 1, further comprising a connecting plate having a recess at least partially defining the first mixing space and openings fluidly coupling the first pump and the second pump to the first mixing space.

8. The spray device of claim 1, further comprising:
a post including:
a plurality of nozzle grooves; and
a plurality of channel-forming protrusions fluidly coupling a first mixing space to a second mixing space.

9. The spray device of claim 1, further comprising a top housing including:
a nozzle opening; and
defining:
a portion of a second mixing space; and
a portion of a nozzle mixing space.

10. The spray device of claim 1, further comprising a cap operable to be coupled to a top housing.

11. The spray device of claim 1, further comprising:
a pump housing coupled between: the liquid container, and an outlet tube of the first pump and an outlet tube of the second pump;
a first pump fitting configured to retain the first pump;
a second pump fitting configured to retain the second pump;
a base operable to connect to a connecting tongue of the liquid container; and
a collar.

12. The spray device of claim 1, further comprising a gasket coupled between the liquid container and a pump housing.

13. The spray device of claim 1, wherein the liquid container further comprises:
a divider separating the first chamber and the second chamber;
an outer housing;
a bottom forming a bottom of the first chamber and the bottom of the second chamber;
a connecting tongue operable to connect to a base of a pump housing.

14. The spray device of claim 1, wherein:
the first pump comprises:
a first inlet tube in fluid communication with the first chamber, wherein the first inlet tube includes a first inlet, a first piston, a first spring, and a first ball valve; and
a first outlet tube including a first outlet fluidly coupled to the first mixing space; and
the second pump comprises:
a second inlet tube in fluid communication with the second chamber, wherein the second inlet tube includes a second inlet, a second piston, a second spring, and a second ball valve; and
a second outlet tube including a second outlet fluidly coupled to the first mixing space.

15. The spray device of claim 1, wherein:
a tip portion of the top housing is configured to spray at an angle ranging from about 0° to about 90° relative to a flow path out of the second mixing space; or
the nozzle is configured to spray a predetermined pattern of liquid including one or more of a hollow cone, a full cone, a spiral cone, a solid stream, a mist, a fog, a flat fan, or combinations thereof.

16. The spray device of claim 1, wherein a viscosity of the first liquid or the second liquid, or a combination thereof is from about 0.1 centipoise (cP) to about 10,000 CP.

17. The spray device of claim 1, wherein:
a volume of the first chamber is from about 1 ml to about 100 ml; or
a volume of the second chamber is from about 1 ml to about 100 ml.

18. The spray device of claim 1, wherein a ratio of a volume of the first chamber a volume of the second chamber is from about 1:10 to about 10:1.

19. The spray device of claim 1, wherein a volume of the first chamber and a volume of the second chamber is substantially equal.

20. The spray device of claim 1, wherein the spray device is operable to spray a liquid amount of from about 0.01 ml to about 5 ml upon each actuation of the first and second pumps.

21. The spray device of claim 1, wherein:
a ratio of a volume of the first mixing space to a volume of the second mixing space is from about 1:10 to about 10:1; or
a ratio of a volume of the first mixing space to a volume of the third mixing space is from about 1000:1 to about 10:1; or
a ratio of a volume of the second mixing space to a volume of the third mixing space is from about 1000:1 to about 10:1.

22. The spray device of claim 1, wherein:
the first pump is operable to aspirate and retain a liquid amount of from about 0.01 ml to about 5 ml; or
the second pump is operable to aspirate and retain a liquid amount of from about 0.01 ml to about 5 ml.

23. The spray device of claim 1, wherein:
a ratio of a volume of: a capacity of the first pump or the second pump, and a volume of a first mixing space or a second mixing space is from about 100:1 to about 1:1; or
a ratio of a volume of: a capacity of the first pump or the second pump, and a volume of a nozzle mixing space is from about 10000:1 to about 10:1.

24. The spray device of claim 1, wherein an exit velocity from the nozzle is from about 6.5 m/s to about 19.5 m/s.

25-67. (canceled)
